(19) United States
(12) Patent Application Publication
(10) Pub. No.: US 2012/0282305 A1
(43) Pub. Date: Nov. 8, 2012

(54) BIOLOGICAL SURFACTANTS FOR CONNECTION TO SILICONE-BASED MATERIALS AND MODULATING LEVELS OF IMMUNOLOGICALLY ACTIVE PROTEINS

(75) Inventors: Xiaolan Lin, Flushing, NY (US); Miriam Rafailovich, Plainview, NY (US)

(73) Assignee: The Research Foundation of State University of New York, Albany, NY (US)

(21) Appl. No.: 13/520,380

(22) PCT Filed: Jan. 7, 2011
(86) PCT No.: PCT/US2011/020494

§ 371 Date: Jul. 3, 2012

Related U.S. Application Data
(60) Provisional application No. 61/293,395, filed on Jan. 8, 2010.

(51) Int. Cl. A61K 38/16; B05D 5/06; B05D 5/00; A61K 9/00; C12N 5/071

(52) U.S. Cl. 424/400; 514/1.1; 435/375; 435/353; 435/352; 435/350; 435/351; 435/363; 435/366; 427/2.3; 427/2.31; 427/2.24; 427/164

(57) ABSTRACT

Biological surfactants connected to surfaces of silicone-based materials are provided. Compositions of electrolytes and a biological surfactant are also provided. Methods for increasing the surface wettability of a silicone-based material by contacting the silicone-based material with a biological surfactant, methods for increasing evaporation from a silicone-based material by contacting a surface of the silicone-based material with a biological surfactant, methods for increasing levels of interleukin-8 during inflammation by contacting a cell with a biological surfactant, and methods for decreasing expression of a biological surfactant by contacting a cell with an siRNA are further provided.

### CROSS-REFERENCE TO RELATED APPLICATIONS

This application claims the benefit of priority of U.S. Provisional Patent Application No. 61/293,395, filed Jan. 8, 2010, the entire contents of which are hereby incorporated by reference.

### STATEMENT REGARDING FEDERALLY SPONSORED RESEARCH OR DEVELOPMENT

This invention was made with government support under Division of Materials Research Project No. 1055789, Sponsor No. 0606387, Award No. 39804 awarded by the National Science Foundation. The government has certain rights in the invention.

### BACKGROUND OF THE DISCLOSURE

The ocular surface is covered by a stratified, squamous and non-keratinizing epithelium that is about five to seven cells thick. The corneal epithelium is open to the environment, and thus is exposed to a variety of biological, chemical, or mechanical insults.

To maintain its proper barrier functions, the healthy corneal epithelium, along with the tear fluid in which it is bathed, forms an intricate defense system ranging from simple rinsing by tear fluid to complex innate or adaptive immune responses. Ultraviolet irradiation (UV) and pathogen invasion represent the most common environment threats to the eye. Ultraviolet radiation is subdivided into three classes, namely UVA, UVB, and UVC. The ocular epithelium as the outermost layer of the eye absorbs a substantial amount of UVB irradiation than any other ocular tissue, therefore acting as a filter to protect lens and retina from UV-induced damage (Ringvold, A., “Corneal epithelium and UV-protection of the eye”, Acta Ophthamol. Scand. 76(2):149-53 (1998)). Exposing to high doses of UVB however, would cause cell shedding to the tear pool (Kolozsvári, L., “UV absorbance of the human cornea in the 240- to 400-nm range”, Invest. Ophthalmol. Vis. Sci. 43(7):2165-8 (2002)). Small amount of cell debris can be simply removed by mechanical rinsing or by tear fluid, but significant cell death in the ocular surface may increase the susceptibility of the eye to pathogen invasions.

In addition to UV irradiation, the ocular surface is also exposed to pathogens (bacteria, viruses, and fungi) on a daily basis. Innate immunity serves as the first line defense against invading pathogens and involves such components as antimicrobial peptides (e.g. β-defensin) and phagocytes (e.g. neutrophils and macrophages) for quick and non-specific clearance of dead cells and foreign objects. Under normal circumstances, the innate immunity is sufficient for protecting the eye from infection. However, in severe cases adaptive immune responses are activated to summon immunologically responsive cells (e.g. T-cells and B-cells) to tackle the challenge. This process takes relatively longer and has more profound consequences. Excessive and prolonged inflammatory process could generate undesirable outcomes including deep wounds and scar tissue formation. Ocular keratitis is an eye condition in which chronic inflammation leaves behind scar tissues in or on the corneal surface, causing blurry vision or even blindness. Therefore, quick onset and resolution of inflammation is vital not only for a timely recovery of ocular function, but also for healing without scarring that is essential for maintaining corneal transparency and visual acuity.

Epithelial injury and pathogens can initiate inflammatory reaction, which is an orchestrated process mediated by a myriad of proinflammatory cytokines such as IL-1α, IL-1β and TNF-α, which subsequently induce the expression of chemokines such as IL-8 in keratinocytes and epithelial cells (Cubitt, C. L., et al., “IL-8 gene expression in cultures of human corneal epithelial cells and keratocytes”, Invest. Ophthalmol. Vis. Sci. 34(11):3199-206 (1993); Elner, V. M., et al., “Human corneal interleukin-8. IL-1 and TNF-induced gene expression and secretion”, Am. J. Pathol. 139(5):977-88 (1991); Kumar, A., et al., “Innate immune response of corneal epithelial cells to Staphylococcus aureus infection: role of peptidoglycan in stimulating proinflammatory cytokine secretion”, Invest. Ophthalmol. Vis. Sci. 45(10):3513-22 (2004); Venza, I.; et al., “Transcriptional regulation of IL-8 by Staphylococcus aureus in human conjunctival cells involves activation of AP-1”, Invest. Ophthalmol. Vis. Sci. 48(1):270-6 (2007)). Interleukin-8, referred to as CXC chemokine ligand 8 (CXCL8) is one of the most potent neutrophil chemoattractants. In the case of corneal inflammation, early augmentation of IL-8 may contribute to timely recruitment of neutrophils from the peripheral regions (e.g. the eyelids) to the avascular cornea (Sloop, G. D., et al. “Acute inflammation of the eyelid and cornea in Staphylococcus keratitis in the rabbit”, Invest. Ophthalmol. Vis. Sci. 40(2):385-91 (1999)). Additionally, IL-8 has been shown to stimulate α-smooth muscle actin production in fibroblasts and to cause wounds to contract and close more rapidly (Feugate, J. E., et al., “The cxc chemokine cCAF stimulates differentiation of fibroblasts into myofibroblasts and accelerates wound closure”, J. Cell. Biol. 156(1):161-72 (2002)). Interleukin-8 is also chemotactic for fibroblasts, accelerating their migration and the deposition of extracellular matrix proteins during wound healing (Kuhlmann, U. C., et al. “Radiation-induced matrix production of lung fibroblasts is regulated by interleukin-8”, Int. J. Radiat. Biol. 85(2):138-43 (2009)). Prompt and tightly regulated IL-8 production has a positive impact on pathogen prevention and wound healing.

An artificial tear fluid is commonly composed of electrolytes, which typically increase the salt content of the natural tear fluid, a nonionic surfactant, which typically lowers the surface tension of the artificial tear fluid and enhances the spreading of the artificial tear fluid over the surface of the cornea, and antimicrobial preservatives. However, most antimicrobial preservatives are toxic to ocular epithelial cells and disrupt the lipid layer of the tear film. This disruption can make the tear film even more unstable and can aggravate the damage caused by inflammation associated with the ocular surface. Other adverse effects of antimicrobial preservatives include disruption of intracellular desmosomes, increases of the corneal permeability, decreases in the activity of lysozyme in the tear fluid, and sensitization with risk for ocular allergy.

Surfactant proteins were originally discovered in alveolar lining of the lung (Phizackerley, P. J., et al., “Hydrophobic proteins of lamellated osmiophilic bodies isolated from pig lung”, Biochem. J. 183(3):731-6 1979). Surfactant protein-B and surfactant protein-C are hydrophobic while surfactant protein-A and surfactant protein-D are hydrophilic. The secondary protein structures of surfactant protein-B and surfactant protein-C contains alpha-helical loops and beta sheets. In contrast, surfactant protein-A and surfactant protein-D are considered mammalian lectins, which are carbohydrate-binding proteins. More specifically, surfactant protein-A and surfactant protein-D are considered “collectins” (or C-type lectins, group III), which is a subgroup of the mammalian lectins. Each collectin monomer contains four distinct domains: 1) a carbohydrate recognition domain (a “CRD” or lectin domain); 2) a neck domain containing a short hydrophobic stretch of amino acids and an amphipathic helix; 3) a collagen-like domain of Gly-X-Y repeats wherein X and Y are often occupied by proline and hydroxyproline; 4) an amino terminal domain containing a cysteine involved in inter-chain disulfide bond formation (Crouch, E. C., “Structure, biologic properties, and expression of surfactant protein D (SP-D)”, Biochim. Biophys. Acta. 1408(2-3):278-89 (1998)). Basic structural units of collectins consist of three monomers oriented in parallel with their collagen-like tails folded into triple helix so as to form a trimer. Surfactant protein-D consists of four of these basic trimer units and positioned tail-to-tail in a cross-like form to form a dodecamer (FIG. 1). Electron microscopy of surfactant protein-D revealed that the rod-like collagen arms (i.e. the collagen-like domain) have a length of 46 nm and the globular carbohydrate recognition domain is 8-9 nm in diameter. Surfactant protein-D molecules can non-covalently associate to form complex multimolecular assemblies that consist of 2-8 dodecamers (Crouch, E., et al., “Molecular structure of pulmonary surfactant protein D (SP-D)”, J. Biol. Chem. 269(25): 17311-9 (1994); Häkansson, K, et al., “Crystal structure of the trimeric alpha-helical coiled-coil and the three lectin domains of human lung surfactant protein D”, Structure 7(3):255-64 (1999)).

All types of surfactant protein (surfactant protein-A, surfactant protein-B, surfactant protein-C and surfactant protein-D) are known to reduce the surface tension of the alveolar membranes in the lung, thereby make breathing easier (Pérez-Gil, J., & Keough, K. M., “Interfacial properties of surfactant proteins”, Biochim. Biophys. Acta. 1408(2-3):203-17 (1998)). The anti-bacterial properties of surfactants are also well-documented (Restrepo, C. I., et al., “Surfactant protein D-stimulates phagocytosis of Pseudomonas aeruginosa by alveolar macrophages”, Am. J. Respir. Cell. Mol. Biol. 21(5):576-85 (1999); Vaandrager, A. B., & van Golde, L. M. “Lung surfactant proteins A and D in innate immune defense”, Biol. Neonate 77(Suppl. 1):9-13 (2000); Wu, H., et al., “Surfactant proteins A and D inhibit the growth of Gram-negative bacteria by increasing membrane permeability”, J. Clin. Invest. 111(10):1589-602 (2003)). In lung alveola, surfactant proteins-A and -D are known to primarily mediate host defense by binding to intruding pathogens (Restrepo, C. I., et al., “Surfactant protein D stimulates phagocytosis of Pseudomonas aeruginosa by alveolar macrophages”, Am. J. Respir. Cell. Mol. Biol. 21(5):576-85 (1999), whereas surfactant proteins-B and -C maintain the low surface tension of a phospholipid monolayer by facilitating the adsorption and spreading of phospholipids at the air/liquid interface (Ding, J., et al., “Effects of lung surfactant proteins, SP-B and SP-C, and palmitic acid on monolayer stability”, Biophys. J. 80(5):2262-72 (2001)). Surfactant proteins have recently been discovered in non-pulmonary tissues such as skin epidermis (Mo, Y. K., et al., “Surfactant protein expression in human skin: evidence and implications”, J. Invest. Dermatol. 127(2):381-6 (2007)), nasal mucus membrane (Vaandrager, A. B., & van Golde, L. M. “Lung surfactant proteins A and D in innate immune defense”, Biol. Neonate 77(Suppl. 1):9-13 (2000); Woodworth, B. A., et al., “Surfactant protein A and D in human sinus mucosa: a preliminary report”, J. Otorhinolaryngol. Relat. Spec. 69(1):57-60 (2007)), and tear fluid (Brauer, L., et al. “Detection and localization of the hydrophobic surfactant proteins B and C in human tear fluid and the human lacrimal system”, Curr. Eye Res. 32(11):931-8 (2007); Brauer, L., et al., “Detection of surfactant proteins A and D in human tear fluid and the human lacrimal system” Invest. Ophthalmol. Vis. Sci. 48(9):3945-53 (2007)). These findings further support the view that surfactant proteins have a positive impact on host defense and lipid barrier stabilization (Wright, J. R., “Immunoregulatory functions of surfactant proteins”, Nat. Rev. Immunol. 5(1):58-68 (2005)). While the epithelia of the lung and the cornea share certain features, the corneal epithelium has unique properties and functions. Therefore, prior to this disclosure, the function, if any, of surfactant proteins in connection with the corneal surface were the matter of speculation.

Even though surfactant proteins-A and -D have long been acknowledged for their active roles in host defense, the mechanisms by which they are involved in an inflammatory reaction are unclear. Specifically, some studies support the view that surfactant proteins-A and -D are proinflammatory (Blau, H., et al., “Secretion of cytokines by rat alveolar epithelial cells: possible regulatory role for SP-A”, Am. J. Physiol. 266(2 Pt. 1):L148-55 (1994); Meloni, F., et al., “Surfactant apoprotein A modulates interleukin-8 and monocyte chemotactic peptide-I production”, Eur. Respir. J. 19(6):1128-35 (2002)), some studies support the view that the proteins are anti-inflammatory (Borron, P., et al., “Surfactant-associated protein A inhibits LPS-induced cytokine and nitric oxide production in vivo”, Am. J. Physiol. Lung Cell. Mol. Physiol. 278(4):L840-7 (2000); Sato, M., et al., “Direct binding of Toll-like receptor 2 to zymosan, and zymosan-induced NF-κB activation and TNF-α secretion are down-regulated by lung collectin surfactant protein A”, J. Immunol. 171(1):417-25 (2003); Snyder, G. D., et al., “Surfactant protein D is expressed and modulates inflammatory responses in human coronary artery smooth muscle cells”, Am. J. Physiol. Heart Circ. Physiol. 294(5):H2053-9 (2008)), while some studies support the view that the proteins are both pro- and anti-inflammatory (Gardai, S. J., et al., “By binding SIRPα or calreticulin/CD91, lung collectins act as dual function surveillance molecules to suppress or enhance inflammation”, Cell 115(I):13-23 (2003); Gold, J. A., et al. “Surfactant protein A modulates the inflammatory response in macrophages during tuberculosis”, Infect. Immun. 72(2):645-50 (2004); Haczkul, A., “Protective role of the lung collectins surfactant protein A and surfactant protein D in airway inflammation”, J. Allergy Clin. Immunol. 122(5):861-79 (2008)).

It is speculated that surfactant proteins-B and -C are an integral part of the corneal epithelium and may serve essential role in maintaining a stable and continuous layer of tear fluid. However, much less is known about their involvement in immune responses, possibly as a result of technical difficulties in characterizing and purifying these small and hydrophobic proteins.

Polydimethysiloxane (“PMDS”, often referred to as silicones) has the following chemical formula:

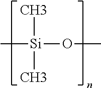

Silicone polymers can be easily transformed into elastomers by a way of cross-linking that allows the formation of chemical bonds between adjacent chains. Silicone elastomers have been widely used in biomedical applications such as medical-grade tubing, transdermal drug delivery patches, implanted prostheses, and contact lenses. Silicone-based materials are considered to be both biocompatible, because they are generally non-toxic and induce only minimal tissue responses, and bio-durable, because they are thermally and chemically stable. These advantageous biophysical properties of silicone-based materials have been attributed to their unique material properties such as high oxygen permeability, flexibility, including a low glass transition temperature, T_{g}, that is typically less than 120° C., and chemical inertness. However, silicone-based materials are hydrophobic in nature owing to their non-polar/hydrophobic methyl (—CH₃) side groups pointing towards the surfaces, and in many cases would require some degree of chemical alteration to allow for adequate compatibility with the host tissue.

### BRIEF SUMMARY OF THE DISCLOSURE

Disclosed is the exposure of a silicone-based material to a preparation containing at least one surfactant protein (SP). Surface deposition of e.g., SP-D, enhances surface wettability of the silicone-based material.

Disclosed is the role of surfactant proteins in immune responses, including the activity of surfactant proteins and the activity of immunologically active proteins in response to a harmful insult. Also disclosed is the role of surfactant proteins in IL-8 production of corneal epithelium triggered by UVB radiation or bacterial peptidoglycan. Further disclosed are the functions of surfactant proteins in an induced inflammatory reaction in corneal epithelial cells.

Disclosed is a silicone-based material and at least one biological surfactant, wherein the surfactant protein is connected to a surface of the silicone-based material.

Disclosed is a method for increasing the surface wettability of a silicone-based material by contacting the silicone-based material with at least one biological surfactant.

Disclosed is a method for increasing evaporation from a silicone-based material by contacting a surface of the silicone-based material with at least one biological surfactant.

Disclosed is an “artificial tears” composition which contains at least one biological surfactant.

Disclosed is a method for increasing levels of interleukin-8 during inflammation by contacting a cell with at least one biological surfactant.

Disclosed is a method for decreasing expression of a biological surfactant by contacting a cell with a siRNA.

### BRIEF DESCRIPTION OF THE FIGURES

FIG. 1. A schematic of the structure of surfactant protein-D.

FIG. 2. The definitions of the drop parameters, including contact angle, contact base diameter, and height.

FIG. 3. Images of water drops during evaporation: (a) a water drop on a control surface and (b) a water drop on a silicone surface exposed to solutions of surfactant protein. The inserted time in each image represents the time after the deposition of water drops onto each surface. The scale bar in each image represents 1 mm.

FIG. 4. (a) Contact angle θ, (b) contact base diameter W, and (c) drop height h for drops of water on a silicone surface exposed to solutions of surfactant protein-B, surfactant protein-C, and surfactant protein-D. (d) Water contact angles of silicone surfaces exposed to solutions of surfactant protein-D.

FIG. 5. (a) Contact angle θ, (b) contact base diameter W, (c) drop height h, (d) surface area A, and (e) drop volume V for two microliter drops of water on a silicone surface exposed to solutions of surfactant protein.

FIG. 6. Roughness analysis of silicone surfaces exposed to a solution of surfactant protein-D and control surfaces by tapping mode AFM. (a) Roughness analysis for a control silicone surface; the scan size was 10 μm. (b) Roughness analysis for a silicone surface exposed to a solution of surfactant protein-D; the scan size was 10 μm.

FIG. 7. (a), (b), and (c): Analysis of thickness of surfactant protein-D connected to three silicone surfaces by tapping mode AFM. The scan size was 10 μm in all cases.

FIG. 8. Tapping mode AFM images of silicone surfaces exposed to a solution of surfactant protein-D and control surfaces. Panels (a) and (b) represent the height maps of a control surface and a silicone surface exposed to a solution of surfactant protein-D, respectively; panels (c) and (d) represent a top view of a control surface and a silicone surface exposed to a solution of surfactant protein-D, respectively; and panels (e) and (f) represent a sectional analysis of a control surface and a silicone surface exposed to a solution of surfactant protein-D, respectively.

FIG. 9. Study of time-dependent release of surfactant protein-D from silicone surfaces.

FIG. 10. Proposed orientations of surfactant protein-D (“SP-D”) molecules on a silicone surface. In these proposed orientations, a single layer of molecules is affixed to the surface in an “end-on” position, with the carbohydrate recognition domain (“CRD”) exposing to the air.

FIG. 11. Possible effects of exposure of a silicone surface to a solution of a surfactant protein to subsequent wetting.

FIG. 12. Effect of surfactant protein-gene silencing on UV- or PGN-induced IL-8 gene transcription and protein secretion. Top panel: Gene silencing of surfactant protein suppressed UV-induced IL-8 production of both protein and mRNA. Bottom panel: Gene silencing of surfactant proteins suppressed PGN-induced IL-8 production of both protein and mRNA. Data are the mean±the standard error of results in three independent experiments and are expressed as a percentage of untreated, non-induced cells transfected with control siRNA. Production of IL-8 was compared between cells transfected with surfactant protein siRNA and those transfected with control siRNA. * indicates a probability less than 0.05 based on Student's two-tailed t-test.

FIG. 13. Effect of rhSP on UV- or PGN-induced IL-8 gene transcription and protein secretion. Top panel: Effect of rhSPs on UV-induced IL-8 production of both protein and mRNA. Bottom panel: Effect of rhSPs on PGN-induced IL-8 production of both protein and mRNA. Data are the mean±the standard error of results in three independent experiments and are expressed as a percentage of untreated/non-induced cells. After exposure to either UV or PGN, production of IL-8 in cells grown in the presence of rhSP was compared to those grown without rhSP. * indicates a probability less than 0.05 based on Student's two-tailed t-test.

FIG. 14. Dose-effect of rhSP on UV- or PGN-induced IL-8 secretion. Top panel: Effect of rhSP on UV-induced IL-8 secretion. Bottom panel: Effect of rhSP on PGN-induced IL-8 secretion. Attached cells were lyzed and assayed for total protein concentrations (BCA, Pierce). Concentrations of secreted IL-8 were normalized for total protein in cell lysates. Data are the mean±the standard of results in three independent experiments and are expressed as percentage of untreated and non-induced cells.

FIG. 15. rhSP restored UV-induced IL-8 gene transcription and protein secretion in surfactant protein siRNA transfected cells. Data are the mean±the standard error of results in three independent experiments and are expressed as a percentage of untreated and un-induced cells transfected with control siRNA.

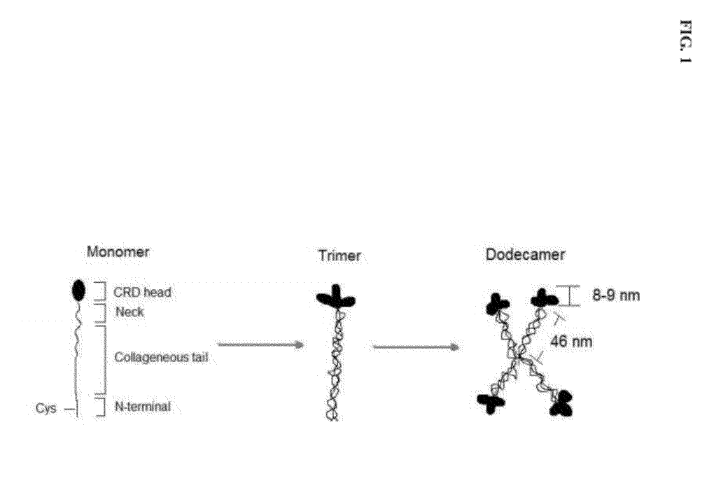

### DETAILED DESCRIPTION OF THE DISCLOSURE

The disclosure provides biological surfactants connected to surfaces of silicone-based materials. By “silicone”, it is meant a polymerized siloxane. In some embodiments, the silicone is polydimethysiloxane (“PDMS”) having the following chemical formula:

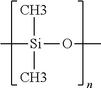

By “silicone-based material”, it is meant an item made substantially from silicone, variants of silicone, or a combination of silicone and one or more variants of silicone. In some embodiments, the item is made from at least about 50% silicone, variants of silicone, or a combination of silicone and one or more variants of silicone, from at least about 60% silicone, variants of silicone, or a combination of silicone and one or more variants of silicone, from at least about 70% silicone, variants of silicone, or a combination of silicone and one or more variants of silicone, from at least about 80% silicone, variants of silicone, or a combination of silicone and one or more variants of silicone, from at least about 90% silicone, variants of silicone, or a combination of silicone and one or more variants of silicone, from at least about 95% silicone, variants of silicone, or a combination of silicone and one or more variants of silicone, from at least about 99% silicone, variants of silicone, or a combination of silicone and one or more variants of silicone, or from 100% silicone, variants of silicone, or a combination of silicone and one or more variants of silicone. In some embodiments, the item is made from 100% silicone.

In some embodiments, the silicone-based material is adapted for medical use. In some embodiments, the silicone-based material is adapted for veterinary use. In some embodiments, the silicone-based material may be capable of being sterilized. In some embodiments, the silicone-based material is medical-grade tubing, a transdermal drug delivery patch, an implanted prosthesis, or a contact lens.

By a “biological surfactant”, it is meant one or more polypeptides independently capable of lowering the surface tension of a liquid. In some embodiments, the biological surfactant is capable of acting as a wetting agent. In some embodiments, the biological surfactant is hydrophobic. In other embodiments, the biological surfactant is hydrophilic. In some embodiments, the biological surfactant is a recombinant protein. In some embodiments, the biological surfactant is a surfactant protein. In some embodiments, the biological surfactant is surfactant protein-A, surfactant protein-B, surfactant protein-C, or surfactant protein-D. In some embodiments, the biological surfactant is made up of more than one surfactant protein species.

In some embodiments, the biological surfactant is in solid form. In some embodiments, the biological surfactant is in suspension. In some embodiments, the biological surfactant is in liquid form. In some embodiments, the biological surfactant is a solute. In some embodiments, the biological surfactant is in the form of an aerosol. In some embodiments, the biological surfactant is in the form of a gaseous phase.

By “connected” in relation to two items, it is meant that the two items are affixed, one to the other, in a non-transitory form. In this disclosure, such a state of affixation may be referred to as a “connection”. In some embodiments, two items are connected by adhesion, i.e. the surface of one item is affixed in a non-transitory form to the surface of the other. In other embodiments, one item is absorbed into the second item. In other embodiments, one item is adsorbed onto the second item.

By “surface”, it is meant a plane of an item that is capable of being contacted by a fluid. In some embodiments, the plane of an item is in contact with a fluid.

By “fluid”, it is meant a substance or combination of substances in liquid or gaseous form. In some embodiments, the fluid is a liquid. In some embodiments, the liquid is miscible with water. In other embodiments, the liquid is not miscible with water. In some embodiments, the liquid is an emulsion. In some embodiments the liquid is a gel, including a hydrogel.

By “recombinant protein”, it is meant a polypeptide produced by a cell that does not naturally produce that polypeptide. In some embodiments, the polypeptide is encoded by a vector within the cell.

By “surface wettability”, it is meant the capacity of a fluid to maintain contact with a solid surface. In some embodiments, the fluid is a liquid. In some embodiments, the liquid is miscible with water. In other embodiments, the liquid is not miscible with water. In some embodiments, the liquid is an emulsion. In some embodiments, the liquid is a gel, including a hydrogel.

By a “preparation”, it is meant a formulation of one or more substances. In some embodiments, the preparation is a solid. In some embodiments, the preparation is a suspension. In some embodiments, the preparation is a liquid. In some embodiments, the preparation is a solution. In some embodiments, the preparation is an aerosol. In some embodiments, the biological surfactant is in the form of a gas.

By “condensing”, it is meant the change of the phase of the substance from gaseous to liquid.

By “depositing”, it is meant the change of the phase of the substance from gaseous to solid.

By “levels” of an immunologically active protein, it is meant the amount of immunologically active protein capable of being detected in a sample. In some embodiments, an increase in the levels of the immunologically active protein is the result of increased quantities of the immunologically active protein. In some embodiments, an increase in the levels of the immunologically active protein is the result of increased activation of the immunologically active protein.

By “inflammation” it is meant a pathological condition evidenced by one or more of heat, pain, redness, swelling, or loss-of-function of a tissue or organ. In some embodiments, the inflammation results from ocular keratitis. In some embodiments, the inflammation results from a mechanical insult, ultraviolet radiation, a pathogen, a molecule derived from a pathogen, a microbial peptide, or an object.

By “ultraviolet radiation”, it is meant electromagnetic radiation having a wavelength of about 100 nanometers to about 400 nanometers. In some embodiments, the ultraviolet radiation has a wavelength of about 100 nanometers to about 280 nanometers, of about 280 nanometers to about 315 nanometers, and of about 315 nanometers to about 400 nanometers. In some embodiments, the radiation is classified as UVA, UVB, and UVC.

In some embodiments, the pathogen is a bacteria, a fungus, or a virus. In some embodiments, the bacteria is a gram-positive bacteria. In some embodiments, the bacteria is a species of Staphylococcus aureus.

In some embodiments, the molecule derived from a pathogen is a molecule derived from a bacterium, a molecule derived from a fungus, or a molecule derived from a virus. In some embodiments, the molecule derived from a bacterium is a peptidoglycan.

In some embodiments, the antimicrobial peptide is β-defensin.

In some embodiments, the object is debris. In some embodiments, the debris is apoptotic debris, i.e. fragments of cells that are undergoing or have undergone apoptotic cell death. In some embodiments, the debris is bacterial debris and fungal debris.

In some embodiments, the cell is a mammalian cell. In some embodiments, the mammalian cell is a mouse cell, a rat cell, a ferret cell, a guinea-pig cell, a rabbit cell, a sheep cell, a goat cell, a pig cell, a cow cell, a dog cell, a cat cell, a monkey cell, a baboon cell, a chimpanzee cell, or a human cell.

By “expression”, it is meant the presence of a molecule on or in association with a cell. In some embodiments, the expression is that of an RNA. In other embodiments, the expression is that of a protein.

By “siRNA”, it is meant a short-interfering ribonucleic acid having a nucleotide sequence, a part of which is identical to the nucleotide sequence of a transcribed strand of a gene of interest.

In some embodiments, the siRNA is “double-stranded”, i.e. made up of two ribonucleic acids that are capable of physical separation. In such embodiments, one ribonucleic acid has a nucleotide sequence a part of which is identical to the nucleotide sequence of a transcribed strand of a gene of interest and the other ribonucleic acid has a nucleotide sequence complementary to at least a part of the nucleotide sequence of the first ribonucleic acid. In some embodiments, the other ribonucleic acid has a nucleotide sequence complementary to at least a part of the nucleotide sequence of the first ribonucleic acid that is also complementary to the untranscribed strand of the gene of interest. In some embodiments, the nucleotide sequence a part of which is identical to the nucleotide sequence of a transcribed strand of a gene of interest is 19-25 nucleotides in length. In other embodiments, the nucleotide sequence complementary to at least a part of the nucleotide sequence of the first ribonucleic acid is 19-25 nucleotides in length. In some embodiments, each of the nucleotide sequence a part of which is identical to the nucleotide sequence of a transcribed strand of a gene of interest and the nucleotide sequence complementary to at least a part of the nucleotide sequence of the first ribonucleic acid is 19-25 nucleotides in length. In some embodiments, the one ribonucleic acid has a nucleotide sequence that is longer than the nucleotide sequence of the other ribonucleic acid. In other embodiments, the other ribonucleic acid has a nucleotide sequence that is longer than the nucleotide sequence of the one ribonucleic acid. In such embodiments, the one ribonucleic acid and the other ribonucleic acid are capable of forming a double-stranded RNA having an “overhang”, i.e. a sequence of nucleotides in the one ribonucleic acid that does not have a complement in the other ribonucleic acid or a sequence of nucleotides in the other ribonucleic acid that does not have a complement in the one ribonucleic acid. In some such embodiments, the overhang is two nucleotides in length. In these embodiments, the overhang is at the 3′ end of the one ribonucleic acid or the other ribonucleic acid.

In some embodiments, the siRNA is “single-stranded”, i.e. made up of a single ribonucleic acid. It is understood that a “single-stranded” siRNA is incapable of physical separation because, unlike double-stranded siRNA, any given “single-stranded” siRNA is a single substance. In some embodiments, the nucleotide sequence of the single-stranded siRNA has a part that is complementary to at least a part of the nucleotide sequence a part of which is identical to the nucleotide sequence of a transcribed strand of a gene of interest. In some embodiments, the nucleotide sequence complementary to at least a part of the nucleotide sequence a part of which is identical to the nucleotide sequence of a transcribed strand of a gene of interest is also complementary to the untranscribed strand of the gene of interest. In some embodiments, the nucleotide sequence a part of which is identical to the nucleotide sequence of a transcribed strand of a gene of interest and the nucleotide sequence a part of which is complementary to at least a part of the nucleotide sequence a part of which is identical to the nucleotide sequence of a transcribed strand of a gene of interest are not separated by another nucleotide sequence.

In other embodiments, the nucleotide sequence a part of which is identical to the nucleotide sequence of a transcribed strand of a gene of interest and the nucleotide sequence a part of which is complementary to at least a part of the nucleotide sequence a part of which is identical to the nucleotide sequence of a transcribed strand of a gene of interest is separated by another nucleotide sequence. In other embodiments, the nucleotide sequence a part of which is identical to the nucleotide sequence of a transcribed strand of a gene of interest is 19-25 nucleotides in length. In some embodiments, the part that is complementary to at least a part of the nucleotide sequence a part of which is identical to the nucleotide sequence of a transcribed strand of a gene of interest is 19-25 nucleotides in length. In some embodiments, each of the nucleotide sequence, a part of which is identical to the nucleotide sequence of a transcribed strand of a gene of interest and the part that is complementary to at least a part of the nucleotide sequence a part of which is identical to the nucleotide sequence of a transcribed strand of a gene of interest, is 19-25 nucleotides in length.

In some embodiments, the single-stranded RNA has an “overhang”, i.e. a sequence of nucleotides does not have a complement in the nucleotide sequence a part of which is identical to the nucleotide sequence of a transcribed strand of a gene of interest or the nucleotide sequence having the part that is complementary to at least a part of the nucleotide sequence a part of which is identical to the nucleotide sequence of a transcribed strand of a gene of interest. In some such embodiments, the overhang is two nucleotides in length. In these embodiments, the overhang is at the 3′ end of the nucleotide sequence.

Disclosed is a silicone-based material and at least one biological surfactant, wherein the biological surfactant is connected to a surface of the silicone-based material. The silicone-based material can be purchased from e.g., Rubber Sheet Roll (Shippensburg, Pa.). The silicone-based material can be mounted onto a glass slide to assist handling.

The biological surfactant can be connected to a surface of the silicone-based material by exposing a surface of the silicone-based material to a preparation containing at least one biological surfactant, incubation of the preparation-exposed surface for a period of time, removal of the preparation, and drying of the silicone-based material.

The biological surfactant can be one or more species of surfactant proteins. The species of surfactant proteins can be surfactant protein-A, surfactant protein-B, surfactant protein-C, or surfactant protein-D or a combination of two or more surfactants. The surfactant protein can be procured from a commercial source such as R&D Systems (Minneapolis, Minn.) or Cusabio Biotech (Wuhan, China), for example. The biological surfactant can be a recombinant protein, i.e. the product of a cell that does not normally produce the protein.

The major constituent of the preparation containing the biological surfactant can be the biological surfactant. Thus a preparation containing the biological surfactant can include at least 51% (w/v or v/v) of biological surfactant, or combinations of biological surfactant. In another embodiment a preparation can include greater than 60%, 70%, 80% or 90% of at least one biological surfactant. Alternatively, preparations containing the biological surfactant can include less than 50% (w/v or v/v) of biological surfactant, or combinations of biological surfactant. In another embodiment, a preparation can include less than 40%, less than 30%, less than 20%, less than 10% and less than 5% of at least one biological surfactant. In other embodiments, preparations containing the biological surfactant can include 50% (w/v or v/v) of biological surfactant, or combinations of biological surfactant.

The preparation containing the biological surfactant can be a solid or semi-solid. The preparation of the biological surfactant can be a powder, a paste, or in the form of a spray. The silicone-based material can be contacted with the biological surfactant by brushing or painting the silicone-based material with a solid or semi-solid preparation of the biological surfactant. The silicone-based material can also be contacted with the biological surfactant by dipping the silicone-based material in a solid or semi-solid preparation of the biological surfactant. Furthermore, the silicone-based material can be contacted with the biological surfactant by spraying the silicone-based material with a preparation of the biological surfactant.

The preparation containing the biological surfactant can be a liquid. The biological surfactant can be a solute of the liquid or suspended in the liquid. The biological surfactant can be in the form of an aerosol, which can be formed from a liquid preparation containing the biological surfactant by, for example, propelling the preparation through a small aperture. The silicone-based material can be contacted with the biological surfactant by brushing or painting the silicone-based material with a liquid preparation containing the biological surfactant. The silicone-based material can also be contacted with the biological surfactant by dipping the silicone-based material in a liquid preparation of the biological surfactant.

The silicone-based material can be a contact lens. The contact lens can aid vision or can be purely decorative. The contact lens can be “hard” or “soft”. The contact lens can also be reusable or disposable.

The liquid preparation containing the biological surfactant can be a liquid suitable for the care and maintenance of a contact lens. The liquid part of the liquid preparation, i.e. the solvent of a solution, the suspendent of a suspension, or the carrier of an aerosol, can be a liquid typically used to care for or maintain contact lenses. The liquid preparation of the contact lens solution can be provided in single-use vessels. The liquid preparation of the contact lens solution can also be sterile.

The liquid preparation containing the biological surfactant can be a solution suitable for connecting a biological surfactant to an eyeglass. The liquid part of the liquid preparation, i.e. the solvent of a solution, the suspendent of a suspension, or the carrier of an aerosol, can be a liquid typically used to care for or maintain eyeglasses, for example an eyeglass cleaner.

The liquid preparation containing the biological surfactant can be a solution suitable for caring for the eyes. The liquid part of the liquid preparation, i.e. the solvent of a solution, the suspendent of a suspension, or the carrier of an aerosol, can be a liquid typically used to care for the eyes, for example “artificial tears”.

The preparation containing the biological surfactant can be a gas. Gaseous preparations containing the biological surfactant can be formed by sublimation, evaporation, or vaporization. The silicone-based material can be contacted with the biological surfactant by condensing a gaseous preparation containing the biological surfactant onto the silicone-based material. The silicone-based material can also be contacted with the biological surfactant by depositing the biological surfactant onto the silicone-based material from a gaseous preparation containing the biological surfactant.

The silicone-based material can be incubated in the presence of the preparation containing the biological surfactant at ambient temperature, which is typically in the range 15-25° C. The silicone-based material can be incubated at body temperature, which is typically 37° C. for humans. The silicone-based material can be incubated in a moist environment to limit evaporation of the preparation containing the biological surfactant. Alternatively, the silicone-based material can be incubated in a dry environment to accelerate evaporation of the preparation containing the biological surfactant.

The preparation containing the biological surfactant can be removed from the silicone-based material by rinsing the silicone-based material with a liquid, such as deionized water, water, or any other suitable liquid. The preparation containing the biological surfactant can be removed from the silicone-based material by dipping the silicone-based material in a liquid. The silicone-based material can be dried at ambient temperature or at body temperature. The silicone-based material can be dried in a dessicator.

In reference to “removing” the preparation containing the biological surfactant, it is understood that not all of the contents of the preparation will necessarily be removed. For example, it is disclosed herein that when a silicone-based material is exposed to a preparation containing a biological surfactant, some of the biological surfactant becomes connected to the silicone-based material.

Disclosed is a method for increasing the surface wettability of a silicone-based material by contacting the silicone-based material with a biological surfactant.

Increases in the surface wettability of or increases in evaporation from a silicone-based material can be ascertained by measuring the dimensions of fluid drops over a time period sufficient for at least some of the fluid drop to evaporate. The dimensions of the fluid drops can be contact angle, contact base, height, surface area, and volume of the fluid drops.

Disclosed is a method for increasing evaporation from a silicone-based material by contacting a surface of the silicone-based material with at least one biological surfactant.

Disclosed are combinations of electrolytes and at least one biological surfactant.

All or some of the electrolytes can be procured independently from e.g. commercial suppliers and combined to constitute “artificial tears”. It is understood that the “artificial tears” can be a commercial preparation including those marketed under the trademarks Aquasite®, Liquifilm®, Refresh®, Systane®, or Visine®. The electrolytes can be of sufficient quality for use in the manufacture of pharmaceuticals. The primary ionic constituents of the electrolytes can be one or more of sodium (Na⁺), potassium (K⁺), calcium (Ca²⁺), magnesium (Mg²⁺), chloride (Cl⁻), hydrogen phosphate (HPO₄₂—), and hydrogen carbonate (HCO₃⁻). It is understood that the overall ionic constituents of the artificial tears will be such that the artificial tears have net ionic charge. The ionic constituents of the electrolytes can be selected to be hypotonic, isotonic, or hypertonic with respect to the natural tear fluid or natural tear film.

The combinations of electrolytes and at least one biological surfactant can further comprise one or more preservative. The preservative can be benzalkonium chloride, cetrimonium chloride, cetrimonium bromide, chlorobutanol, ethylenediaminetetraacetic acid, polyquarternium-42, polyhexamethylene biguanide, silver sulfate, sodium perborate, or thiomersal. The preservatives can be of sufficient quality for use in the manufacture of pharmaceuticals.

The combinations can be sterilized using an autoclave, filtration, radiation or can be procured sterile. The disclosure provides pharmaceutical compositions containing electrolytes and one or more biological surfactant and pharmaceutical compositions containing electrolytes, one or more biological surfactant, and a preservative.

Disclosed is an improved preparation of electrolytes, wherein the improvement is the presence of at least one biological surfactant in the preparation. Also disclosed in an improved preparation of electrolytes and preservatives, wherein the improvement is the presence of at least one biological surfactant in the preparation.

The biological surfactant can be one or more species of surfactant proteins. The species of surfactant proteins can be surfactant protein-A, surfactant protein-B, surfactant protein-C, or surfactant protein-D. The surfactant protein can be procured from a commercial source such as R&D Systems (Minneapolis, Minn.) or Cusabio Biotech (Wuhan, China). The biological surfactant can be a recombinant protein, i.e. the product of a cell that does not normally produce the protein.

A biological surfactant can be added to the “artificial tears”, whether the “artificial tears” are constituted or procured. The concentration of the biological surfactant can be 1-1,000 ng/ml. In some embodiments, the concentration of the biological surfactant can be 62.5, 125, 250, 500, or 1,000 ng/ml.

Disclosed is a method for increasing levels of interleukin-8 during inflammation by contacting a cell with a biological surfactant.

Levels of IL-8 can be determined by immunofluoresence, flow cytometry, enzyme-linked immunosorbent assay (“ELISA”), immunoblotting, chromatography, or bioassay. It is understood that a biological surfactant increases the levels of interleukin-8 during inflammation if the level of IL-8 in a sample from a cell contacted with a biological surfactant is greater than the level of IL-8 in a sample from a comparable cell that has not been contacted with a biological surfactant.

The inflammation can be induced by the patient, induced experimentally, or induced by a stimulus. The inflammation can be in the ocular area. The ocular area can be the interior of the eyelid, the exterior of the eyelid, or the orbit. The inflammation can be induced by the patient by rubbing the ocular area or by contacting the ocular area with an object such as a digit. The inflammation can be induced experimentally by contacting the cell with a substance known to cause inflammation. The stimulus can be a mechanical insult, ultraviolet radiation, a pathogen, a molecule derived from a pathogen, a microbial peptide, and an object.

The mechanical insult can be result from physical contact of the cell or the ocular area.

The ultraviolet radiation can have a wavelength of about 100 nm to about 280 nm, i.e. “UVC”, about 280 nm to about 315 nm, i.e. “UVB”, or about 315 nanometers to about 400 nanometers, i.e. “UVA”. The source of the ultraviolet radiation can be the sun or a source of illumination known to produce ultraviolet radiation.

The pathogen can be a bacterium, fungus, or a virus. The pathogen can be a pathogen known to infect the ocular region. The pathogen can be a gram-positive bacterium. The gram-positive bacterium can be a species of Staphylococcus aureus.

The molecule derived from a pathogen is selected from a group consisting of a molecule derived from a bacterium, a molecule derived from a fungus, and molecule derived from a virus. The pathogen can be a pathogen known to infect the ocular region. The pathogen can be a gram-positive bacterium. The gram-positive bacterium can be a species of Staphylococcus aureus. The molecule derived from a pathogen can be peptidoglycan. The peptidoglycan can be procured from commercial sources (e.g., Sigma-Aldrich, St. Louis, Mo.) or be purified from bacteria. The source of peptidoglycan purified from bacteria can be bacterial cell walls.

The antimicrobial peptide can be β-defensin.

The object can be debris. The debris can be apoptotic debris, bacterial debris, or fungal debris. The apoptotic debris can be prepared from mammalian cells undergoing apoptosis. A mammalian cell can be induced to undergo apoptosis by contacting the mammalian cell with a substance known to induce apoptosis or exposing the mammalian cell to a stimulus known to induce apoptosis.

The cell can be a cell of the eye. The cell of the eye can be a cell of the ocular surface. The cell can be in the ocular area. The ocular area can be the interior of the eyelid, the exterior of the eyelid, or the orbit. The cell of the eye can be a cell of the corneal epithelium. The cell of the eye can be in an intact subject, i.e. present in a subject. The subject can be a mouse, a rat, a ferret, a guinea-pig, a rabbit, a sheep, a goat, a pig, a cow, a dog, a cat, a monkey, a baboon, a chimpanzee, or a human.

Disclosed is a method for decreasing expression of a biological surfactant by contacting a cell with an siRNA.

The siRNA can be procured from a commercial supplier (Santa Cruz Biotech, Santa Cruz, Calif.). The siRNA procured from a commercial supplier can be a pool of three target-specific 19-25 nucleotide-long double stranded RNA molecules with 2-nt 3′ overhangs on each end. The cells are transfected with the siRNA procured from a commercial supplier as the supplier recommends.

The siRNA can also be synthesized. The synthesis can be performed using an automated oligonucleotide sequencer or can be performed in vivo using transfected cells.

The siRNA can be single-stranded or double-stranded. If a single given “siRNA” species is double-stranded, i.e. made up of two ribonucleic acids that are capable of physical separation, a double-stranded region of the gene of interest can be selected such that one ribonucleic acid and the other ribonucleic acid have complementary sequences and that these complementary sequences are identical to a double-stranded region of the gene of interest. The length of these complementary sequences can be 19-25 nucleotides. The length of the one ribonucleic acid can be greater that the length of the other ribonucleic acid such that there are two nucleotides at the 3′ end of the one ribonucleic acid. The length of the other ribonucleic acid can be greater that the length of the one ribonucleic acid such that there are two nucleotides at the 3′ end of the other ribonucleic acid. The double-stranded siRNA can be introduced into the cell using microinjection, electroporation, vector-mediated transfection, or liposome-mediated transfection.

The double-stranded siRNA can be made up of a single double-stranded siRNA species or multiple double-stranded siRNA species.

The individual ribonucleic acids in the siRNA can be “single-stranded”, i.e. a single given “siRNA” species is made up of a single ribonucleic acid. If the siRNA is single-stranded, a double-stranded region of the gene of interest can be selected such that the ribonucleic acid has a self-complementary sequence and that this self-complementary sequence is identical to a double-stranded region of the gene of interest. The length of this complementary sequence can be 19-25 nucleotides. The length of the one ribonucleic acid can be greater than the length of the other ribonucleic acid such that there are two nucleotides at the 3′ end of the one ribonucleic acid. The nucleotide sequence a part of which is identical to the nucleotide sequence of a transcribed strand of a gene of interest and the nucleotide sequence a part of which is complementary to at least a part of the nucleotide sequence a part of which is identical to the nucleotide sequence of a transcribed strand of a gene of interest can be separated by another nucleotide sequence or may not be so separated. The length of the ribonucleic acid can be such that that there are two nucleotides at the 3′ end of the ribonucleic acid that do not have a complement in the self-complementary region. The single-stranded siRNA can be introduced into the cell using microinjection, electroporation, vector-mediated transfection, or liposome-mediated transfection.

The single-stranded siRNA can be made up of a single single-stranded siRNA species or multiple single-stranded siRNA species.

The gene of interest can be a gene encoding a surfactant protein. The gene encoding the surfactant protein can be a gene encoding surfactant protein-A, surfactant protein-B, surfactant protein-C, or surfactant protein-D.

The nucleotides that form the nucleic acids of the siRNA can be substituted ribonucleic acids.

### EXAMPLES

First Set of Materials and Methods

Silicone Rubber Samples

Transparent silicone rubber sheet with thickness of 0.5 mm was purchased from Rubber Sheet Roll (Shippensburg, Pa.). The silicone sheet has adhesive plastic backings on both sides that are peeled off upon testing. To prepare silicone samples, the sheet was cut into pieces of approximately 1.5 cm×1.5 cm and mounted onto a glass slide.

Protein Adsorption

Recombinant surfactant protein-D (Catalog No. 1920-SP-050, R&D Systems, Minneapolis, Minn.) and surfactant protein-B (Cusabio Biotech, Wuhan, China) was prepared in 0.01 M phosphate-buffered saline having a pH of 7.4 (“PBS”) of concentrations of 10 μg/ml and 1 μg/ml, respectively. Under ambient conditions, 150 μl of PBS or PBS containing a surfactant protein was placed onto the surface of a silicone sample. The preparations were then incubated for six hours at room temperature. After incubation, the silicone samples were rinsed twice with Millipore-Q water to remove excessive or loosely affixed proteins. The silicone samples were then air dried for approximately fifteen minutes before any further testing.

Contact Angle Measurement

The wettability of a silicone surface treated with PBS or PBS containing a surfactant protein was evaluated by measuring the contact angle formed drops of 2 μl of water and the surface by using a CAM 200 Optical Contact Angle Meter (KSV instruments Ltd., Helsinki, Finland).

In order to disregard the effects of gravity, the contact radii of the drops should be less than 2.8 mm as determined by the following formula for capillary length:

κ⁻¹=(γ_{LV}/ρg)^{1/2}

where γ_{LV} is the vapor-liquid interfacial tension, ρ is the liquid density and g is the acceleration due to gravity. The range of contact radii for the droplets studied was less than 1.5 mm. Therefore, the water droplet is considered to be sufficiently small and therefore gravity flattening effect can be ignored.

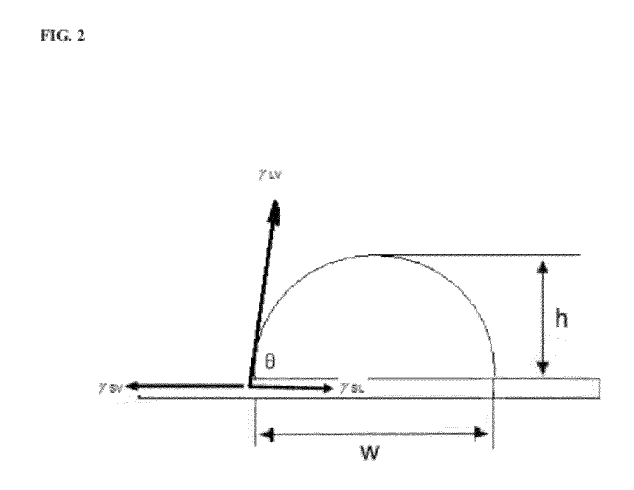

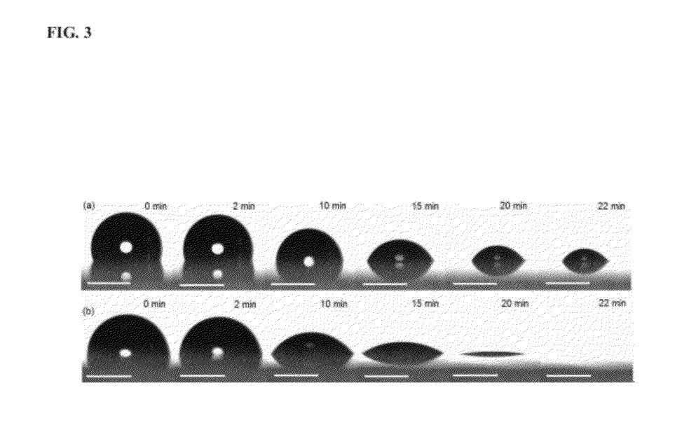

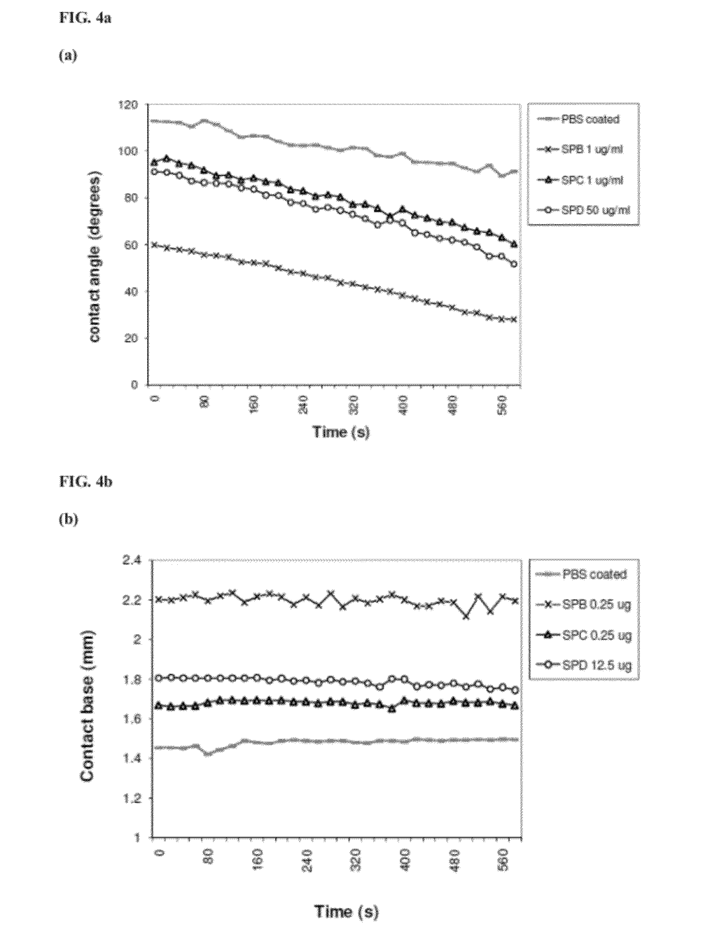

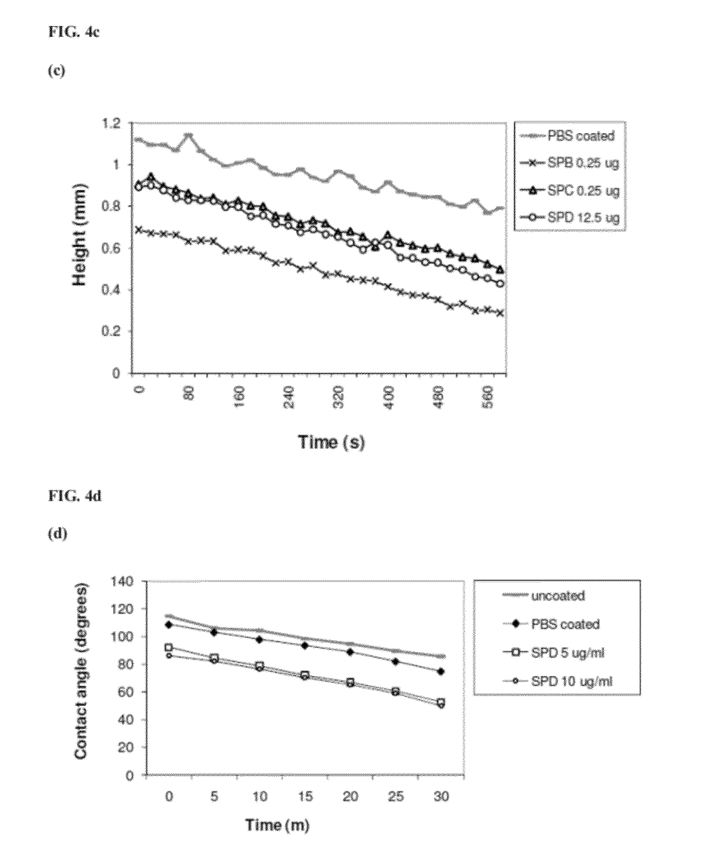

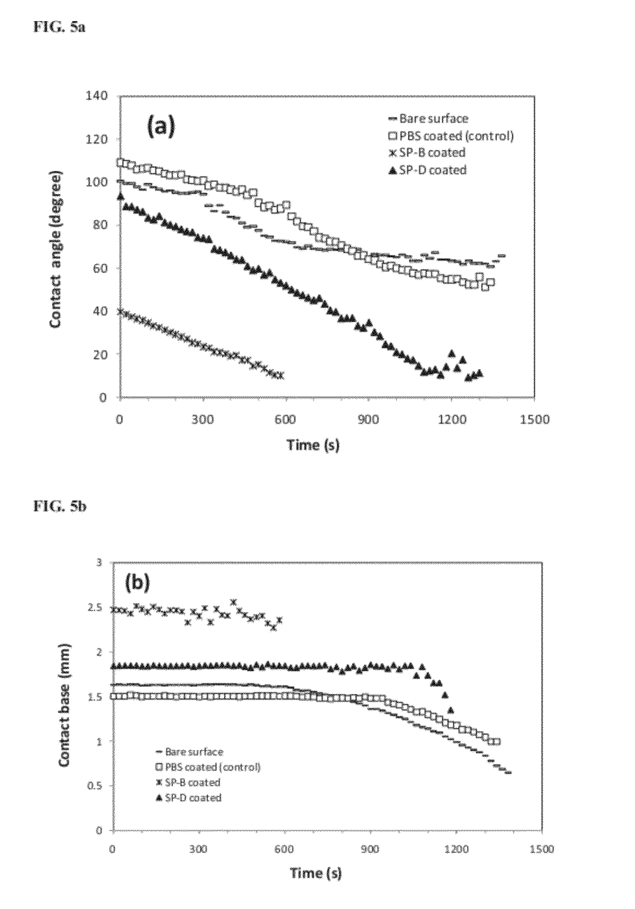

Water drops were mounted on the silicone surfaces with a micropipette. Images of the water drop at a rate of one frame per twenty seconds were recorded and stored. The definitions of the drop parameters such as contact angle, contact base diameter, and height are described in FIG. 2. Using the CAM200 software, a curve was fitted to the drop parameters using the Young-Laplace equation. The contact angle was then determined from the slope of the contour line at the three-phase boundary point.

The equilibrium at a solid (S) and vapor (V) triple line is described by Young's equation:

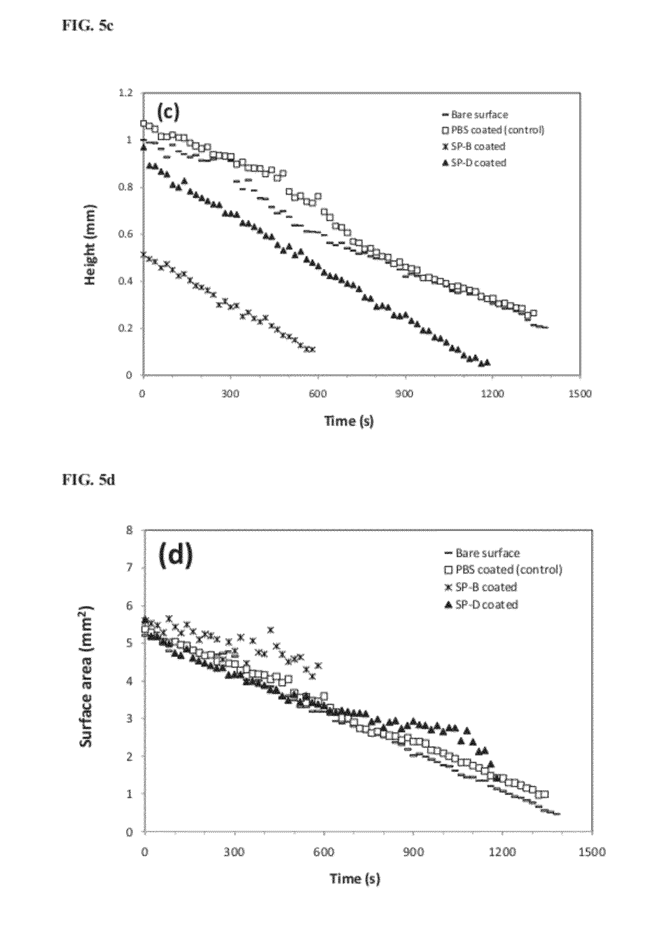

γ_{SV}=γ_{SL}+γ_{LV} cos θ₀ Equation 1

where γ_{SV}, γ_{SL} and γ_{LV} represent surface tensions for the solid/vapor, solid/liquid, and liquid/vapor interfaces, respectively, and θ₀ is the equilibrium contact angle between the tangent planes to the S/L and L/V boundaries at the three phase line or triple line. The contact angle as given by Young's equation is a static and equilibrium angle. However, during its motion toward an equilibrium shape, a liquid droplet spans a range of dynamic contact angles. Long-term behavior of water drops on the surfaces was evaluated by measuring the evolution of their contact angles and contact base diameters on each surface for up to twenty-five minutes or until the droplets disappeared. At least three droplets were observed during their evaporation on each sample and showed good statistical consistency. The evaporation curves set forth in FIG. 5 were chosen as typical for each sample.

Topographic Feature Imaging by Atomic Force Microscopy (AFM)

The silicone samples were mounted on glass slides and imaged with a Nanoscope IIIa instrument (Digital Instruments, Santa Barbara, Calif.) using tapping mode in air. Tips (PPP-NCHR, Nanosensor) that are 125 μm long with spring constants of 42 N/m and resonance frequencies of 330 kHz were used.

Tapping mode AFM is a technique in which the imaging probe is vertically oscillated near the resonant frequency of the cantilever. Electro-mechanical feedback maintains the oscillation at constant amplitude during scanning. The tip intermittently touches or “taps” the surface. The main advantage of tapping mode is the elimination of lateral shearing force that is present in the contact mode, thus reducing the possibility of smearing the protein molecules on the surface. Images were collected at a scan rate of 1.0 Hz and a scan size of 10 μm.

Kinetics of Protein Release by Fluorescence Microscopy

Protein desorption from the silicone surfaces exposed to solutions containing a surfactant protein was examined by fluorescence microscopy (Olympus BX51, Center Valley, Pa.). Silicone surfaces exposed to solutions containing a surfactant protein were placed into a washing chamber containing a PBS solution and incubated at room temperature for 0, 1, 2, 3, 4 or hours with gentle shaking. At specific time intervals, three samples were taken out of the chamber and rinsed with PBS. Where a silicone surface had been exposed to solutions containing a surfactant protein, 0.2 μg/ml of biotinylated anti-surfactant protein-D antibody (BAF1920, R&D Systems) was then applied to the silicone surface. After a one hour incubation, excessive antibody was removed by rinsing with PBS. A 2 mg/ml solution of Texas Red (A2348, Sigma-Aldrich, St. Louis, Mo.) of 2 mg/ml was then applied and incubated for 30-60 min. Texas Red is a highly fluorescent conjugate of avidin and sulforhodamine 101 that fluoresces at about 595 nm and has an emission maximum at about 615 nm. After labeling, the silicone surfaces were rinsed again and allowed to air dry for 15 minutes. Fluorescently-labeled proteins retained on the surfaces were subsequently imaged by fluorescence microscopy. Protein-Texas Red complexes were enumerated and percentages of retained protein were plotted against incubation time to give an estimation of the protein release rate.

First Set of Results

Wetting Behavior of Silicone Surfaces Exposed to Solutions Containing a Surfactant Protein

The water drop contact angles on silicone surfaces exposed to solutions containing a surfactant protein were lower than that of the control surfaces while contact bases remained constant over much of the evaporation time (˜25 minutes) (FIG. 5). On silicone surfaces exposed to a solution containing surfactant protein-B, the contact angle decreased 60 in one hundred and twenty seconds from an initial contact angle of 40°. On silicone surfaces exposed to a solution containing surfactant protein-D, the contact angle decreased 11 in one hundred and twenty seconds from an initial contact angle of 94°. In contrast, on control surfaces the contact angle only decreased 3° in one hundred and twenty seconds from an initial contact angle of 109°.

In summary, the dynamic contact angle decreased more on the surfaces exposed to solutions containing surfactant proteins than on the control surfaces in the same time period. The presence of the surfactant protein in the solutions to which the surfaces had been exposed the only experimental difference between the surfaces exposed to solutions containing surfactant proteins and the control surfaces. Therefore, the decrease in dynamic contact angle over time suggests that the presence of the surfactant protein in the solutions to which the surfaces had been exposed increased the wettability of the silicone surfaces.

The contact angle and contact base plots (FIGS. 5a and 5b) suggest that evaporation of water drops on the silicone surfaces exposed to solutions containing surfactant proteins and the control surfaces evolved in two distinct stages: in the first stage, the base of the drop stayed constant while the contact angle and the height of the water drop decreased linearly and simultaneously. This stage lasted for a long period of time during the evaporation (15 min on the control surface, 7 min on the surface exposed to a solution containing surfactant protein-B, and 18 min on the surface exposed to a solution containing surfactant protein-D). At the end of this stage, the base abruptly decreased but the angle and the height of the drop remained relatively fixed. This stage was rather short and lasted until the drop disappeared (7 min on the control surface, 3 min on the surface exposed to a solution containing surfactant protein-B, and 2 min on the surface exposed to a solution containing surfactant protein-D). By assuming the spherical cap model, the volume, V(t), and the surface area, A(t), of a water drop were calculated using equations 2 and 3, respectively:

$$\begin{matrix}V ( t ) = \frac{π {W}^{3} ( 2 - 3 cos θ + {cos}^{3} θ )}{24 {sin}^{3} θ} & Equation 2 \\ A ( t ) = \frac{π {W}^{2}}{2 ( 1 + cos θ )} & Equation 3\end{matrix}$$

Therefore, evaporation flux, J(t), that is defined as the mass loss per unit time per unit surface area of the water drops can be derived using equation 4:

$$J ( t ) = \frac{{V}_{t - 1} - {V}_{t}}{{A}_{t - 1}} \qquad Equation 4$$

Owing to the high scattering of evaporation flux data, evaporation rates (mass loss over unit time) were calculated to evaluate the evaporation processes of the water drops on silicone surfaces. The slopes of the linear fit of the volume plots (FIG. 5e) for a surface exposed to a solution containing surfactant protein-B, a surface exposed to a solution containing surfactant protein-D, and a control surface were determined to be 0.0017, 0.0013, and 0.0012 mg/second, respectively. Therefore, water drops on surfaces exposed to a solution containing surfactant protein-B and surfaces exposed to a solution containing surfactant protein-D evaporated faster than those on the control surfaces.

Surface Topology by Atomic Force Microscopy

Images of surfaces exposed to a solution containing surfactant protein-D and control surfaces were procured and characterized in air by AFM with the tapping mode. The measured AFM images were analyzed for surface roughness. The section analysis was conducted to depict sectional profiles of protein molecules affixed to the surfaces. Quantitative measurements of the root mean square (RMS) surface roughness were determined using 10 μm×10 μm scans. The RMS roughness is defined as the height fluctuation in a given area:

$$Rms = \sqrt{{\sum }_{j = 1}^{N} {\sum }_{i = 1}^{N} \frac{{( {Z}_{ave} - {Z}_{ij} )}^{2}}{{N}^{2}}} \qquad Equation 5$$

where Zₐᵥₑ is the average value, Zᵢⱼ is the surface height, and N is the number of sections respectively. The number of sections analyzed was 512. The RMS roughness measurements for three control surfaces and three surfaces exposed to a solution containing surfactant protein-D were 17.6±0.26 nm and 12.2±0.31 nm respectively. Therefore, surfaces exposed to a solution containing surfactant protein-D appeared to be slightly smoother than control surfaces.

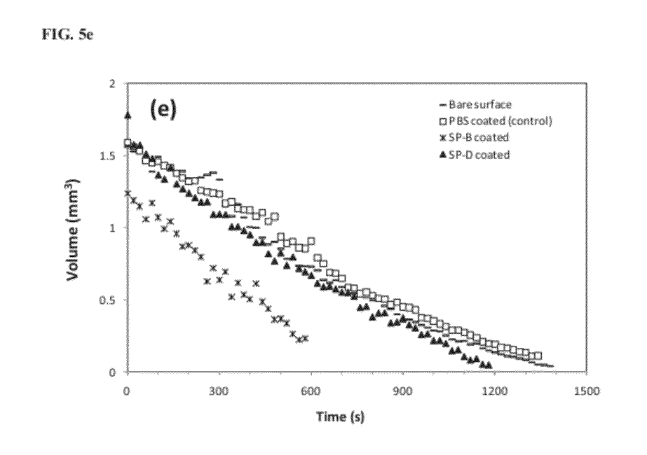

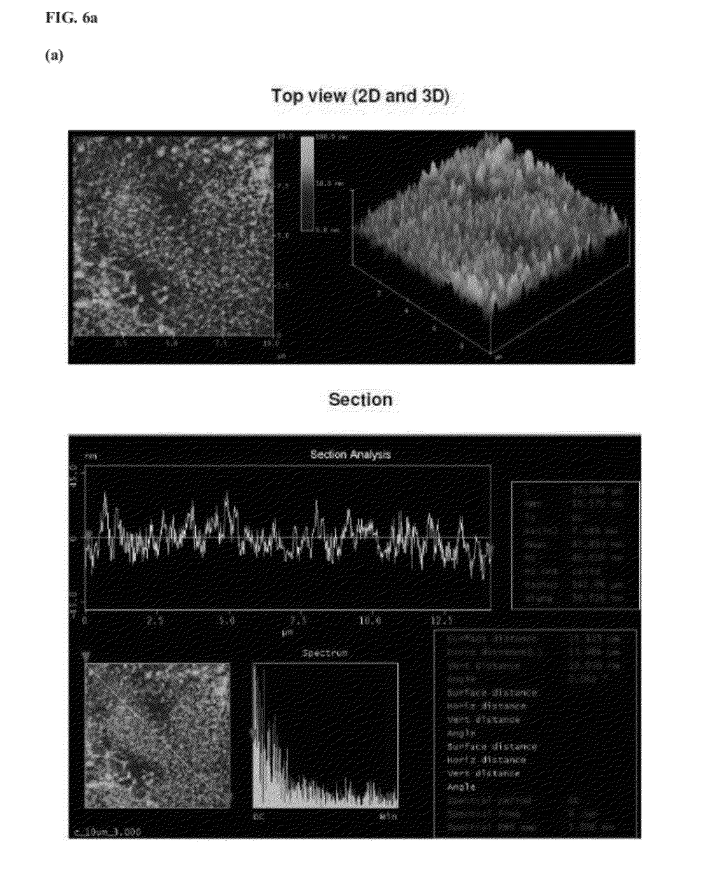

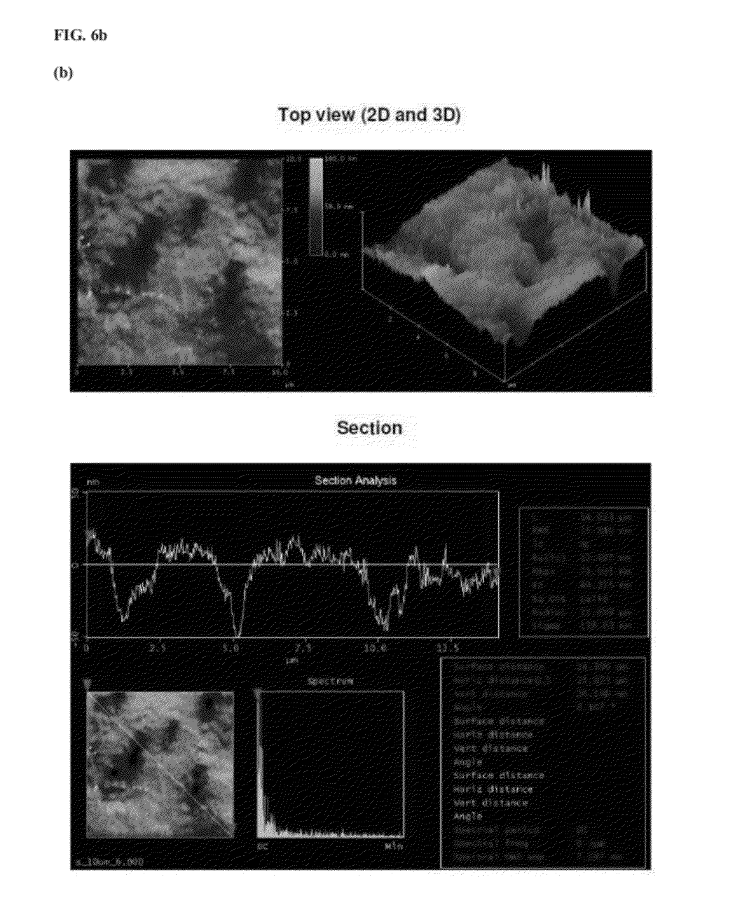

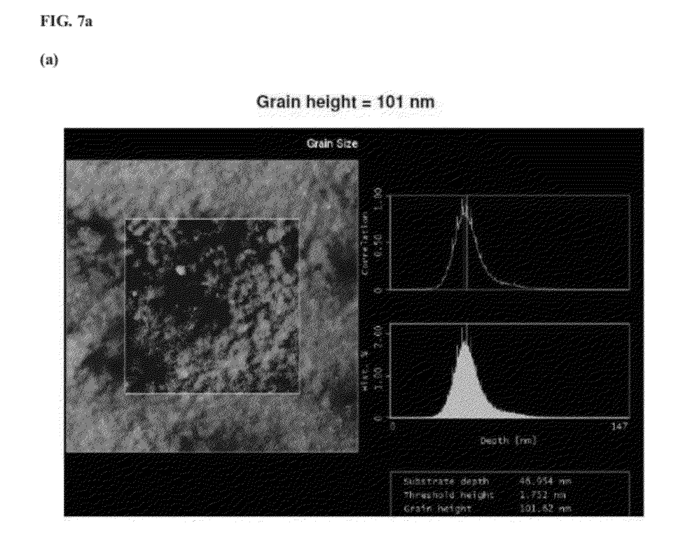

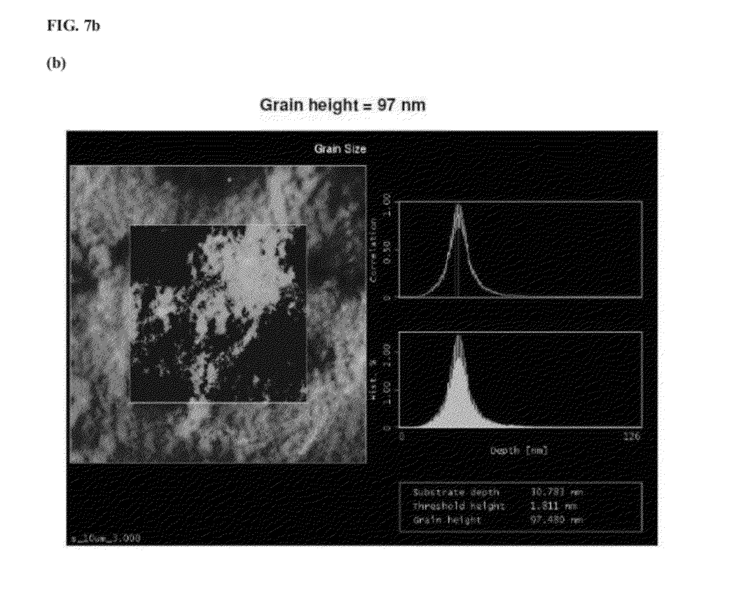

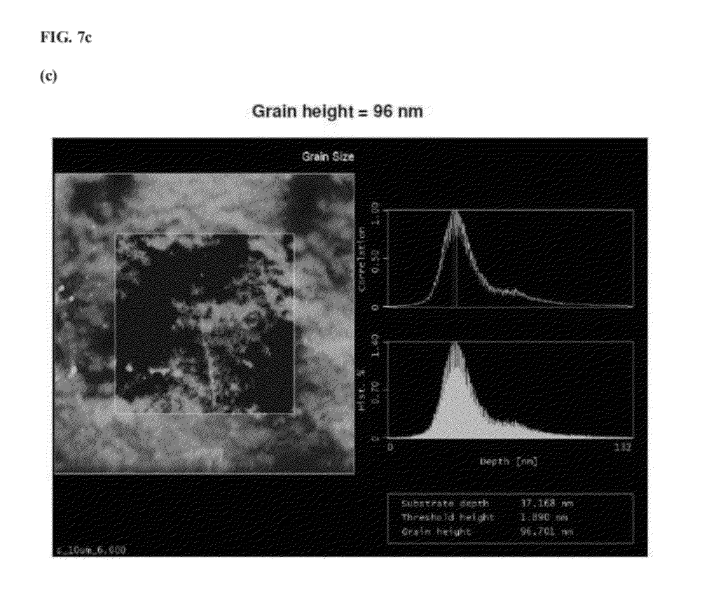

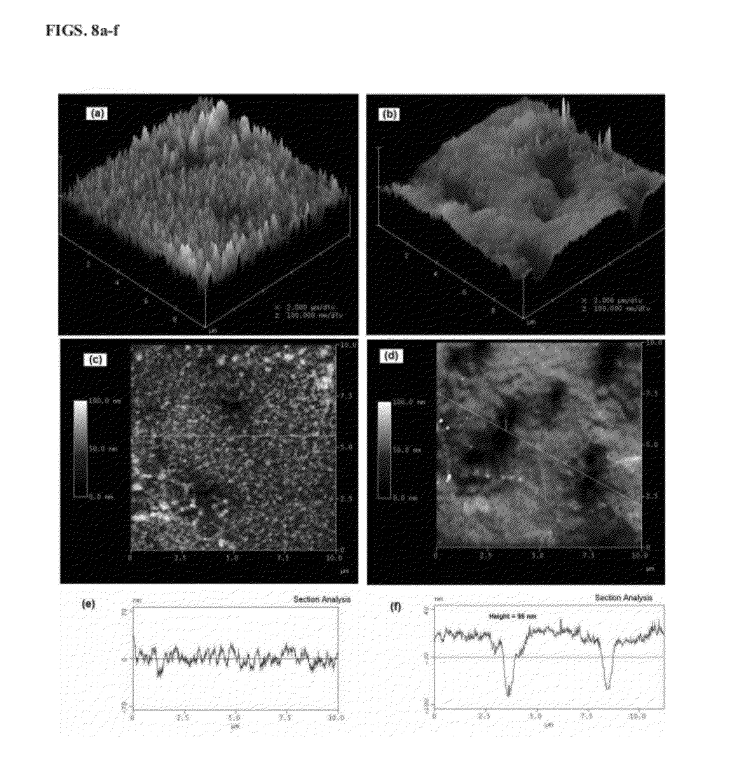

The atomic force microscopy images (FIGS. 6-8) revealed that surfaces exposed to a solution containing surfactant protein-D at a concentration of 10 g/ml were covered with many small compact protein clusters. Sectional analysis of these surfaces revealed that the thickness of the protein coating was 99.6±0.03 nm, which corresponds to the height of a surfactant protein-D dodecamer/multimer molecule in an “end-on” position. As indicated in FIG. 10, the height of a surfactant protein-D dodecamer/multimer molecule in an “end-on” position is less than or equal to 114 nm and varies depending on the angle formed between extended collagen arms.

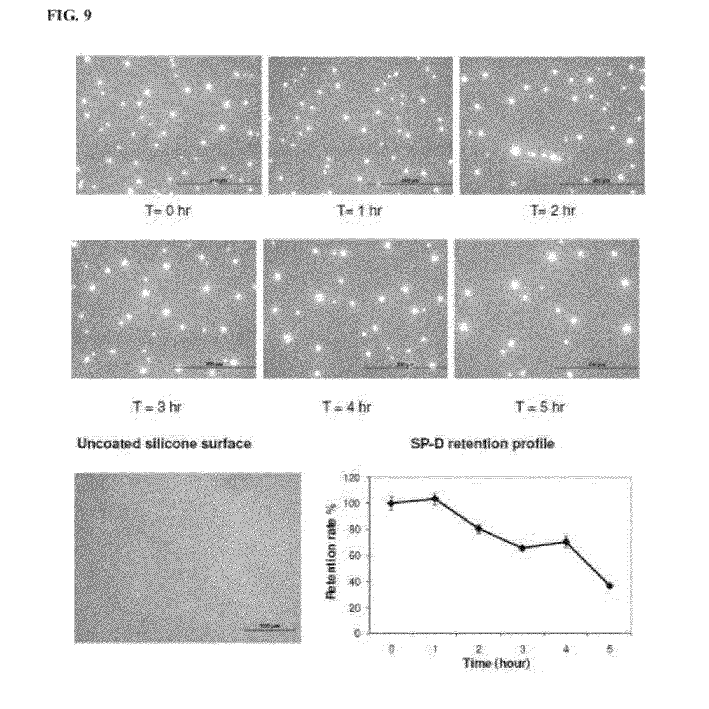

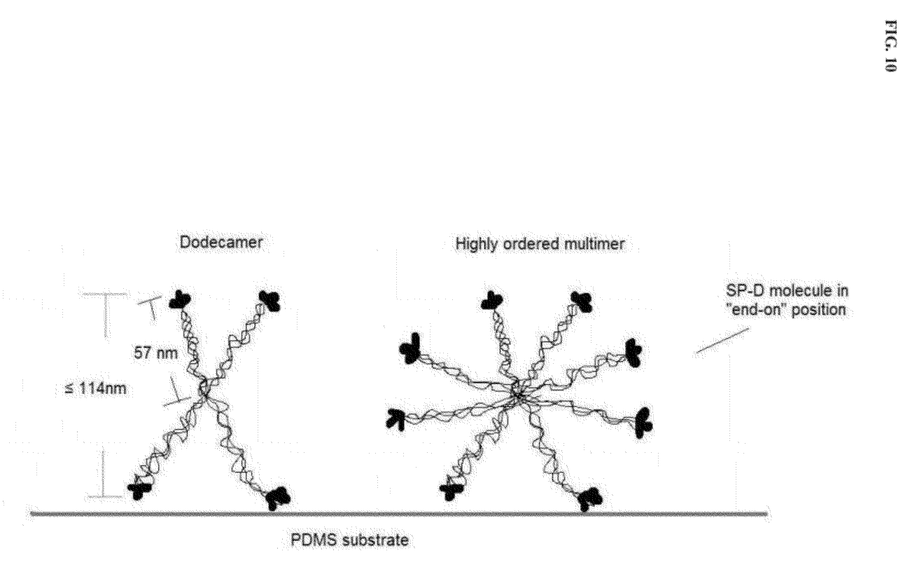

Without limitation to any particular theory, it is thought that wetting is governed by molecular interaction in the outermost surface layer of a few angstroms and so the forces dictating the wetting behavior originate from the outermost surface groups. Furthermore, it is thought that the molecules tend to arrange themselves in the surface layer in such a way that their interfacial tension with the surrounding phase is minimized. Taking surfactant protein-D as an example, and without limitation to any particular theory, the disclosures herein suggest that surfactant proteins in solution expose their hydrophilic moieties, i.e. their collagen-like domains, at their interface with the fluid. The disclosures herein further suggest that surfactant proteins tend to concentrate at the water-air interface, orienting part of their hydrophobic moieties, i.e. their carbohydrate recognition domains, towards the air. The disclosures herein therefore suggest that silicone surfaces exposed to solutions containing a surfactant protein that are subsequently dried have an outwardly-oriented hydrophobic layer of surfactant proteins. Without limitation to any particular theory, the disclosures herein recognize that the surfactant proteins form a coat on the silicone surfaces that initially appears to be hydrophobic that then becomes hydrophilic when exposed to water (FIG. 11).

Without limitation to any particular theory, it is thought that protein affixation to silicone is by adhesion and occurs through hydrophobic interactions owing to the presence of the dominant surface functional group —CH₃ in the silicone. It is proposed that adsorbed surfactant protein-D can take either the “end-on” position with the characteristic height of 114-nm, corresponding to the length of two adjoining collagen arms, or the “side-on” position with the characteristic height of 9-nm, corresponding to the diameter of the globular carbohydrate recognition domain for a monolayer of proteins (FIG. 10). The disclosure that thickness of the protein coating corresponds to the height of a surfactant protein-D dodecamer in the “end-on” position suggests that surfactant protein-D dodecamers or higher-ordered multimers interact with the methyl groups of the silicone monomers through their carbohydrate recognition domains CRD domains with the rigid collagen-like arms arranged towards the air. Although protein molecules may assume the “side-on” position with multiple layers of molecules stacking on top of each other, it is disclosed herein that single-layered protein molecules in the “end-on” position allows for hydrophobic groups to be more densely packed upon exposure to air. This arrangement is considered more energetically preferable than the multiple layers of proteins in the “side-in” position.

This disclosure demonstrates that silicone surfaces exposed to solutions of surfactant proteins had improved wettability evidenced by contact angle measurements compared with control surfaces. Atomic force microscopic image analysis indicated that silicone surfaces exposed to solutions of surfactant protein-D may be covered with a single layer of surfactant protein-D molecules. Without being limited to any particular theory, exposure of silicone surfaces to solutions of at least one surfactant protein is a simple and effective approach to improving surface wettability of silicone-based biomaterials.

Second Set of Materials and Methods

Cell Culture

HCE-2, an adenovirus SV40 immortalized corneal epithelial cell line (CRL-11 135, American Type Culture Collection, Manassas, Va.) was cultured according to the supplier's recommendations. Upon thawing, cells were plated in extracellular matrix gel (Sigma) pre-coated flasks and maintained in keratinocyte serum free medium (“KSFM”, Gibco) containing 0.05 mg/ml bovine pituitary extract, 5 ng/ml human recombinant epidermal growth factor, 500 ng/ml hydrocortisone and 0.005 mg/ml bovine insulin at 37° C. in a 5% CO₂ humidified atmosphere. Medium was renewed twice weekly until cells were ˜80% confluent. Sub-confluent cells were detached from the flask using 0.05% w/v Trypsin and 0.53 mM EDTA, centrifuged at 8000 g for ten minutes, re-suspended and subsequently seeded in a multi-well cell culture plate at an initial density of 20,000 cells/cm². Cells were switched to keratinocyte basal medium (“KBM”, Gibco) twenty-four hours before treatment. To support the function of surfactant proteins, culture medium used for treatment was supplemented with 0.5 mM calcium chloride.

Small Interfering RNA-Mediated Gene Silencing

Commercial small interfering RNA (“siRNA”) for surfactant protein-A, surfactant protein-B, surfactant protein-C, and surfactant protein-D (Santa Cruz Biotech, Santa Cruz, Calif.) is a pool of three target-specific 19-25 nucleotide-long double stranded RNA molecules with 2-nt 3′ overhangs on each end. After entering the cells, siRNA is thought to cause cleavage of target mRNA leading to the inhibition of target gene expression.

Cells were transfected with siRNA according to the manufacturer's recommendations. Healthy and sub-confluent cells were cultured in serum and antibiotic-free normal growth medium containing a mixture of siRNA duplexes and a lipid-based transfection reagent (Santa Cruz) for five to seven hours, followed by additional twenty-four hours culture in further diluted transfection mixture. Control cells were handled in the same manner except that they were transfected with siRNA having a scrambled nucleotide sequence that will not lead to the specific degradation of any known cellular mRNA. Cells were subjected to specific treatment within twenty-four to seventy-two hours after transfection.

Exposure of Cells to UVB Radiation

A UV box equipped with two UVB fluorescent tubes (UBL FSX 24T12/UVB-HO, National Biological Corp., Beachwood, Ohio) was used as light source for irradiation. Irradiance was measured by a radiometer (IL-1700, UV Process Supply, Chicago, Ill.) that equipped with the necessary probe components include detectors, filters, and input optics. Prior to UV irradiation, sub-confluent cells were switched into Hanks Buffered Saline Solution (HBSS). Cell culture plate covers were replaced with Reynolds 914 film to allow better penetration of UV radiation. Cells were irradiated at an intensity of 0.24 mW/cm² for one hundred and twenty-five seconds to achieve a dose of 30 mJ/cm². After exposure, HBSS was aspirated and cells were cultured in normal medium with or without recombinant surfactant proteins. After cultured for additional twenty-four hours, the supernatant was harvested and the cells were rinsed with ice-cold phosphate buffered saline (PBS). The supernatant and cells were assayed immediately or stored at −70° C. until further analysis. Unexposed control plates were handled identically to the other plates except that they received no UV irradiation.

Interleukin-8 Induction Assay

To examine IL-8 induction by UV radiation, HCE-2 cells were exposed to 30 mJ/cm² of UVB as previously described. Cells were then cultured in medium with or without recombinant human surfactant protein (“rhSP”).

To examine IL-8 induction by PGN (Cat. No. 77140, Sigma Aldrich, St. Louis, Mo.), PGN (Cat. No. 77140, Sigma Aldrich, St. Louis, Mo.) was suspended in culture medium to a concentration of 20 μg/ml, bath-sonicated for twenty minutes and added to HCE-2 cells. Cells were cultured with the PGN suspension with or without rhSP for twenty-four hours. The supernatant was subsequently collected and analyzed for IL-8 secretion with a commercial ELISA kit (R&D Systems, Minneapolis, Minn.) according to manufacturer's instructions. Cells were rinsed with ice-cold PBS and were immediately processed for RT-PCR” or stored at −70° C. until further analysis.

Recombinant Human (rh) Surfactant Proteins

Purified, non-tagged recombinant surfactant proteins having the sequences of full-length matured proteins were used in the study (Cusabio Biotech, Wuhan, China). The sizes of rhSP-A, rhSP-B, rhSP-C, and rhSP-D as 25 kDa, 9 kDa, 4 kDa, and 40 kDa respectively were confirmed by SDS-PAGE analysis. These values were quite consistent with that of the respective native mature proteins.

Isolation of RNA and Taqman® Real-Time PCR

Total RNA from the cells was extracted using Qiagen RNeasy Mini kit according to the manufacturer's instructions. The concentration and purity of the RNA preparations were assessed by measuring the absorbance at 260 nm and 280 nm by a spectrophotometer (NanoDrop ND 8000; Bioscience, San Luis Obispo, Calif.). Reverse transcription of 1-2 μg total RNA was performed at 37° C. for two hours and 85° C. for five minutes using a ABI High Capacity cDNA reverse transcription kit (Applied Biosystems, Foster City, Calif.). Ten microliters of first strand cDNA product was adjusted to a volume of 25 μl with a preparation of Taqman Universal PCR Master Mix, Taqman primer, probe sets for a control gene GUSB (beta glucuronidase), and probe sets for the gene encoding IL-8 (ABI Taqman assay no. Hs99999034_m1). Quantitative PCR was performed by using ABI StepOne Plus real-time PCR equipment under the following conditions: fifty cycles at 95° C. for fifteen seconds and 60° C. for one minute. The oligonucleotide sequences and the specific fluorescence-labeled DNA probes were selected to span exon junctions of the target genes. All PCR reactions were performed in triplicate. The comparative Cₜ method was used to quantify transcripts, and the expression level was normalized to that of the human housekeeping gene GUSB or GAPDH. Normalizing the results to the GUSB expression provided results identical to those obtained using GAPDH.

Statistical Analysis

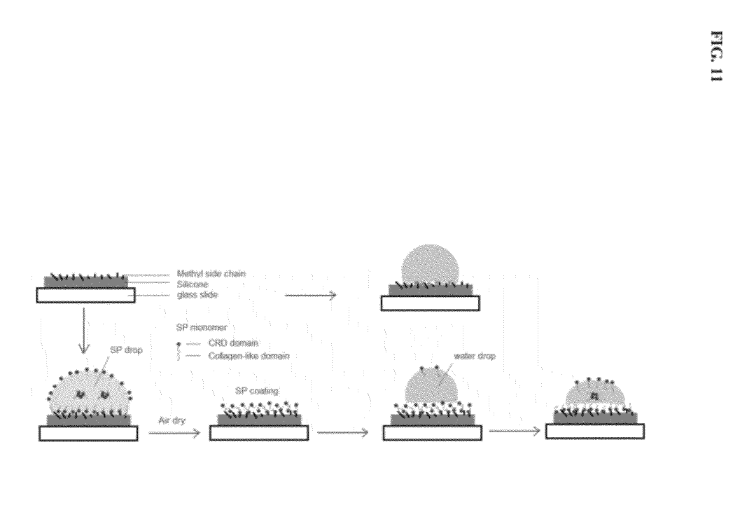

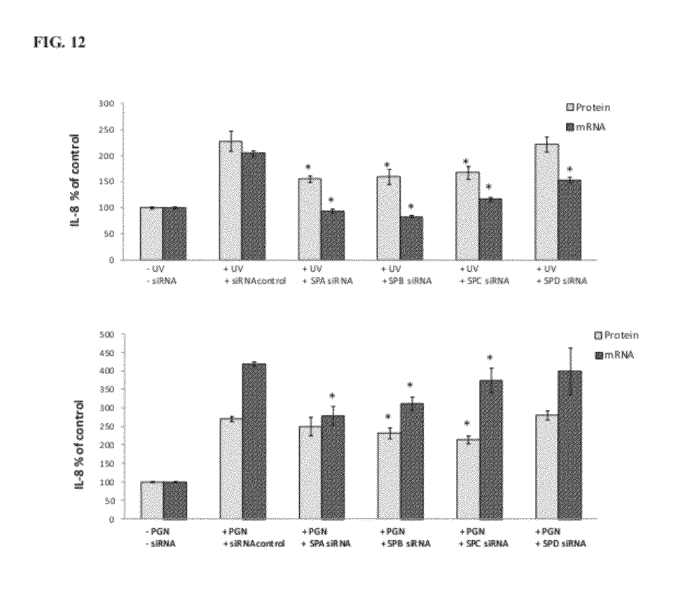

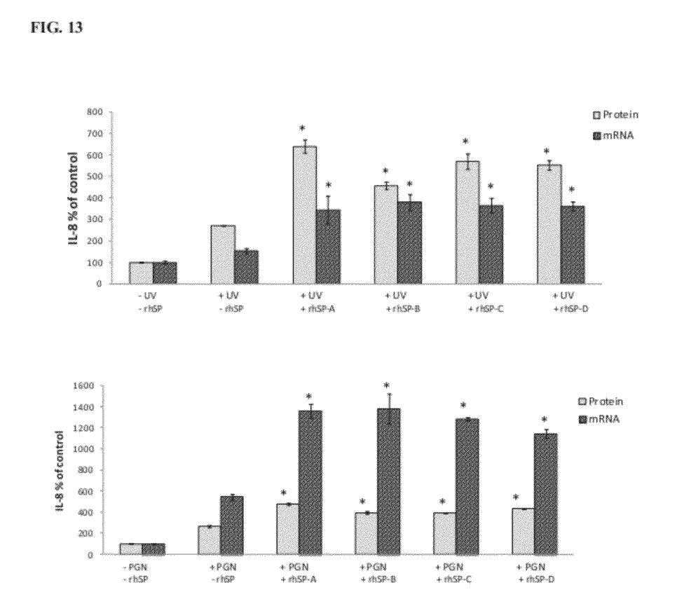

At least three wells were used for each group of samples in all experiments, which were repeated twice. Data for IL-8 secretion/expression studies are expressed as percentages of untreated control±standard error. Untreated controls were cells cultured in normal medium without inducers or testing compounds of any kind. All other data are expressed as means±standard error. The various assay conditions were compared using Student's t test, probability values less than 0.05 were considered to be statistically significant and marked with * in FIGS. 12 and 13.

Second Set of Results

siRNA-Mediated Surfactant Protein Gene Silencing Suppressed UV- and PGN-Induced IL-8 mRNA and Protein Expression

The effect of inhibition of surfactant protein gene expression on UV- or PGN-induction of IL-8 was examined. Cells were transfected with siRNAs targeting surfactant protein-A, surfactant protein-B, surfactant protein-C, or surfactant protein-D. Subsequently, the cells were exposed to either 30 mJ/cm² of UVB radiation or 20 μg/ml of PGN. After twenty-four hours, supernatant and cells were collected for IL-8 secretion and transcription quantification.

Ultraviolet radiation and PGN elicited levels of IL-8 gene transcription and IL-8 secretion at least two-fold those of control cells. The induction was suppressed by transfection with siRNA targeting surfactant protein-A, surfactant protein-B, surfactant protein-C, and surfactant protein-D. Notably, transfection with siRNA targeting of surfactant protein-A and surfactant protein-D had the strongest and weakest suppressive effects on IL-8 production respectively (FIG. 12). Protein expression and gene transcription levels of surfactant proteins were decreased by 30-40% in silenced cells as evaluated by ELISA and PCR analysis, respectively.

rhSP-Enhanced UV- or PGN-Induced IL-8 Production

The effect of recombinant surfactant proteins on UV- and PGN-induced on IL-8 induction of in HCE-2 cells HCE-2 cells were exposed to either 30 mJ/cm² of UV radiation or 20 μg/ml of PGN. Subsequently, the cells were grown in medium containing 50 ng/ml of rhSP-A, rhSP-B, rhSP-C, or rhSP-D for twenty-four hours. The supernatant and cells were subsequently collected for 11-8 protein and mRNA analysis.

The data indicate that all types of rhSP significantly boosted IL-8 protein secretion and mRNA levels in UV- or PGN-induced cells. Gene transcription of IL-8 in PGN-induced cells was prominently stimulated by rhSP (FIG. 13). A time-course study revealed that the IL-8 boosting effect of rhSPs persisted for at least five days following both UV and PGN exposure even though cells were incubated with rhSP only during the first twenty-four hours and regular medium were replaced every other day thereafter.

Dose Effect of rhSP on UV/PGN-Induced IL-8 Secretion

Cells were exposed to 30 mJ/cm² of UV or 20 μg/ml of PGN and subsequently grown in culture medium for twenty-four hours to which rhSP of various concentrations had been added. The IL-8 concentration supernatant was determined for IL-8 concentration.

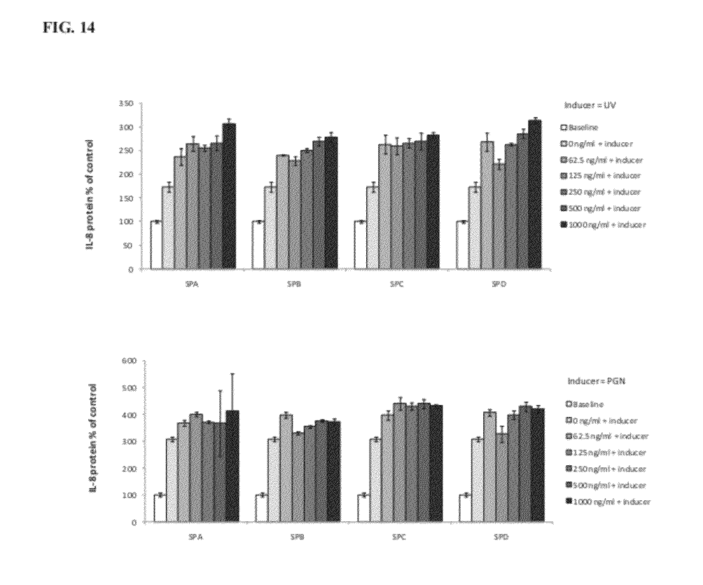

It was determined that rhSPs facilitated UV radiation and PGN in inducing IL-8 at all concentrations tested (FIG. 14). Notably, rhSP-B or rhSP-D of a lower dosage (e.g. 62.5 ng/ml) resulted in an unexpectedly higher induction and this occurred in UV- and PGN-induced cells.

rhSPs Restored UV-Induced IL-8 Gene Transcription and Protein Secretion in Surfactant Protein siRNA Transfected Cells

HCE-2 cells were transfected with a cocktail of siRNAs to simultaneously inhibit the expression of all four types of surfactant protein. Surfactant protein-silenced cells were then exposed to 30 mJ/cm² of UV radiation, followed by twenty-four hours incubation in the presence of 50 ng/ml of rhSP-A, rhSP-B, rhSP-C, or rhSP-D. The supernant was analyzed for IL-8 secretion. Cells were lyzed and assayed for total protein concentrations (BCA, Pierce). IL-8 concentration was normalized for total protein in cell lysates.

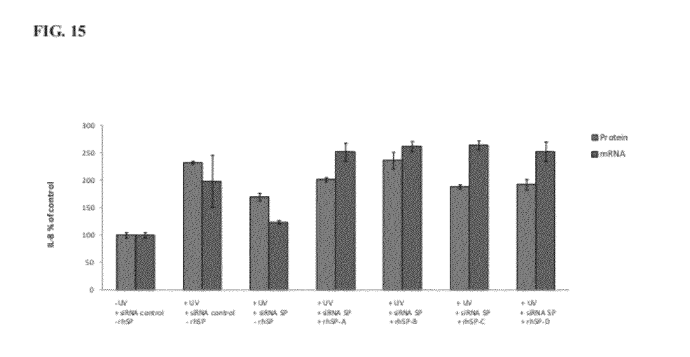

Transfection with surfactant protein siRNA drastically reduced IL-8 gene transcription and protein secretion in UV-exposed cells, whereas surfactant protein supplementation was able to reverse this effect (FIG. 15).

Second Discussion

Surfactant protein-A, surfactant protein-B, surfactant protein-C, and surfactant protein-D augmented the IL-8-inducing effects of UV radiation and PGN in HCE-2 cells. The HCE-2 cell line is an immortalized human corneal epithelial cell line that has been widely used as a model for human corneal epithelial research as it retains morphological, biochemical and functional characteristics of primary corneal epithelia cells. Ultraviolet radiation and PGN represent the most common environment insults to the eye and are known to induce IL-8 via MAP kinase signaling pathways (Azghani, A. O., et al., “Pseudomonas aeruginosa elastase stimulates ERK signaling pathway and enhances IL-8 production by alveolar epithelial cells in culture”, Inflamm. Res. 51(10):506-10 (2002); Kumar, A., et al., “Innate immune response of corneal epithelial cells to Staphylococcus aureus infection: role of peptidoglycan in stimulating proinflammatory cytokine secretion”, Invest. Ophthalmol. Vis. Sci. 45(10):3513-22 (2004); Sun, Y., et al., “Staphylococcus aureus-induced corneal inflammation is dependent on Toll-like receptor 2 and myeloid differentiation factor 88”, Infect. Immun. 74(9):5325-32 (2006)). Ultraviolet radiation causes cell shedding into the tear fluid, and PGN is recognized by cells as intruding pathogens. Objects such as apoptotic cell debris or microbial debris can initiate inflammatory responses by binding to cell surface receptors. Interleukin-8 released as a result of an inflammatory reaction attracts neutrophils and lymphocytes to the inflamed sites to eliminate invading pathogens and halt their spread. For quick recovery of ocular functions, productions of cytokines and chemokines are tightly controlled in time and space to ensure a proper resolution of inflammation. This disclosure recognizes that surfactant proteins are active mediators in an induced inflammatory response that act by boosting IL-8 and support the view that surfactant proteins are proinflammatory when harmful objects are present.

The collectin proteins surfactant protein-A and surfactant protein-D were recently proposed to have dual functions in an inflammatory reaction (Gardai, S. J., et al., “By binding SIRPot or calreticulin/CD91 lung collectins act as dual function surveillance molecules to suppress or enhance inflammation”, Cell 115(1):13-23 (2003); Gold, J. A., et al. “Surfactant protein A modulates the inflammatory response in macrophages during tuberculosis”, Infect. Immun. 72(2):645-50 (2004)). According to this theory, surfactant protein-A and surfactant protein-D exhibit both inflammatory and anti-inflammatory functions, depending on whether their CRD domains are occupied with foreign objects. When the CRDs of surfactant protein-A and surfactant protein-D are bound to cell debris or microorganisms, their collagenous tails interact with calreticulin/CD91 receptors to stimulate phagocytosis and proinflammatory cytokines. It is well known that surfactant protein-A and surfactant protein-D fight bacteria, fungi, and viruses by increasing the permeability of the microbial cell membrane (McCormack, F. X., et al., “Macrophage-independent fungicidal action of the pulmonary collectins”, J. Biol. Chem. 278(38):36250-6 (2003); Wu, H., et al., “Surfactant proteins A and D inhibit the growth of Gram-negative bacteria by increasing membrane permeability”, J. Clin. Invest. 111(10): 1589-602 (2003)). Further, surfactant protein-A has been shown to mediate the phagocytosis of the monocytes by binding to both bacteria and the Clq receptor on monocytes (Geertsma, M. F., et al., “Binding of surfactant protein A to Clq receptors mediates phagocytosis of Staphylococcus aureus by monocytes”, Am. J. Physiol. 267(5 Pt. 1):L578-84 (1994). On the other hand, the resting/un-stimulated conditions the CRDs of surfactant protein-A and surfactant protein-D bind to Toll-like receptors (TLR) or signal inhibitory regulatory protein alpha (SIRPα). This interaction induces downstream inhibition of mitogen-activated protein kinase (MAPK) mediated NF-κB transcription factor, leading to the inhibition of proinflammatory cytokines (Wang, Q., et al., “Micrococci and peptidoglycan activate TLR2->MyD88->IRAK->TRAF->NIK->IKK->NF-κB signal transduction pathway that induces transcription of interleukin-8”, Infect. Immun. 69(4):2270-6 (2001)). According to the above theory, surfactant protein-A and surfactant protein-D serve a protective role by keeping inflammation in check under normal circumstances and become fully engaged in an inflammation when cells sense a threat. This disclosure partially supports this theory. Notably, surfactant protein-A and surfactant protein-D have rather different effects on IL-8 production, suggesting that these proteins may be functionally complementary. Indeed, despite their structural similarity, surfactant protein-A and surfactant protein-D are quite different in biochemical properties which would likely confer distinctive functional properties (Chaby, R., et al. “Interactions between LPS and lung surfactant proteins”, J. Endotoxin Res. 11(3): 181-5 (2005); Crouch, E. C., “Structure, biologic properties, and expression of surfactant protein D (SP-D)”, Biochim. Biophys. Acta. 1408(2-3):278-89 (1998); Kingma, P. S., & Whitsett, J. A., “In defense of the lung: surfactant protein A and surfactant protein D”, Curr. Opin. Pharmacol. 6(3):277-83 (2006)).

This disclosure is the first to provide a link between surfactant protein-B and surfactant protein-C with cytokine modulation in the corneal epithelium. Surfactant protein-B (native or synthetic) has been shown to have anti-microbial activity by selectively lysing bacterial membranes (Ryan, M. A., et al., “Antimicrobial activity of native and synthetic surfactant protein B peptides”, J. Immunol. 176(1):416-25 (2006). Surfactant protein-C, but not surfactant protein-B, has been shown to interact with membrane-bound CD14, a receptor for lipopolysaccharide, suggesting surfactant protein-C may have immunological role during inflammatory processes (Augusto, L. A., et al., “Interaction of pulmonary surfactant protein C with CD14 and lipopolysaccharide”, Infect. Immun. 71(1):61-7 (2003)). However, experimental evidence linking surfactant protein-C to cytokine regulation was still lacking. The present disclosure expands the roles of these hydrophobic surfactant proteins, which have been traditionally thought to only reduce surface tension in lung alveoli.

siRNA provides an effective tool in studying the function of a protein or gene. In this disclosure, inhibition of surfactant protein expression caused a suppressive effect on IL-8 production in corneal epithelial cells, suggesting that surfactant protein depletion may contribute to immune-suppression, which is manifested by reduced or insufficient cytokine production in inflammation. Deficiency in immune responses would greatly increase the chance of ocular infection. Many factors are known to down-regulate surfactant proteins, including TNF-α (Bachurski, C. J., et al., “Tumor necrosis factor-alpha inhibits surfactant protein C gene transcription”, J. Biol. Chem. 270(33):19402-7 (1995); Wispé, J. R., et al., “Tumor necrosis factor-alpha inhibits expression of pulmonary surfactant protein”, J. Clin. Invest. 86(6): 1954-60 (1990)), reactive oxygen species (ROS), nitric oxide synthase-2 (NOS2) (Baron, R. M., et al., “Nitric oxide synthase-2 down-regulates surfactant protein-B expression and enhances endotoxin-induced lung injury in mice”, FASEB J. 18(11):1276-8 (2004), 12-O-tetradecanoyl-phorbol-13-acetate (TPA) (Pryhuber, G. S., et al., “Phorbol ester inhibits surfactant protein SP-A and SP-B expression”, J. Biol. Chem. 265(34):20822-8 (1990)) and hydrogen peroxide (H₂O₂) (Merritt, T. A., et al., “Reduction of the surface-tension-lowering ability of surfactant after exposure to hypochlorous acid”, Biochem. J. 295 (Pt. 1): 19-22 (1993)). Without wishing to be limited to any particular theory, this disclosure also suggests that up-regulation of surfactant protein or supplementation of rhSP can restore an impaired immune function. Factors that up-regulate surfactant proteins include keratinocyte growth factor (KGF); (Sugahara, K., et al., “Keratinocyte growth factor increases mRNAs for SP-A and SP-B in adult rat alveolar type II cells in culture”, Am. J. Physiol. 269(3 Pt. I):L344-50 (1995)), retinoic acid (Bogue, C. W., et al., “Retinoic acid increases surfactant protein mRNA in fetal rat lung in culture”, Am. J. Physiol. 271(5 Pt. 1):L862-8 (1996)), and dexamethasone (Ballard, P. L., et al., “Transcriptional regulation of human pulmonary surfactant proteins SP-B and SP-C by glucocorticoids”, Am. J. Respir. Cell Mol. Biol. 14(6):599-607 (1996)). This disclosure demonstrates that surfactant proteins are associated with modulation of IL-8 activity associated with corneal epithelial cells exposed to UV radiation or PGN. As they possess antimicrobial activity, surfactant proteins in accordance with the present disclosure can be used as an alternative to conventional antibiotics in treating ocular infections or chronic wounds because these “natural antibiotics” will not only prevent the growth of drug-resistant microbes but also expedite the wound healing process by promoting immune responses against infectious agents.

1. An article of manufacture comprising a silicone-based material and a biological surfactant, wherein the biological surfactant is connected to a surface of the silicone-based material.

2. The article of claim 1, wherein the silicone-based material is selected from the group consisting of medical-grade tubing, a transdermal drug delivery patch, an implanted prosthesis and a contact lens.

3. The article of claim 2, wherein the silicone-based material is a contact lens.

4. The article of claim 1, wherein the biological surfactant is selected from the group consisting of surfactant protein-A, surfactant protein-B, surfactant protein-C and surfactant protein-D.

5. The article of claim 1, wherein the biological surfactant is a recombinant protein.

6. The article of claim 1, wherein the surfactant protein is connected to the surface by adhesion.

7. A method for increasing the surface wettability of a silicone-based material, comprising the step of contacting the silicone-based material with a biological surfactant.

8. The method of claim 7, wherein the surfactant protein is selected from the group consisting of surfactant protein-A, surfactant protein-B, surfactant protein-C, and surfactant protein-D.

9. The method of claim 7, wherein the surfactant protein is a recombinant protein.

10. The method of claim 7, wherein the silicone-based material is selected from the group consisting of medical-grade tubing, a transdermal drug delivery patch, an implanted prosthesis and a contact lens.

11. The method of claim 7, wherein the silicone-based material is a contact lens.

12. The method of claim 7, wherein the biological surfactant is in solid form.

13. The method of claim 7, wherein the biological surfactant is in suspension.

14. The method of claim 7, wherein the biological surfactant is a solute.

15. The method of claim 7, wherein the biological surfactant is in the form of an aerosol.

16. The method of claim 7, wherein the biological surfactant is in a gaseous phase.

17. The method of claim 7, wherein the step of contacting comprises brushing the silicone-based material with a preparation of the biological surfactant.

18. The method of claim 7, wherein the step of contacting comprises dipping the silicone-based material in a preparation of the biological surfactant.

19. The method of claim 17, wherein the preparation of the biological surfactant is selected from the group consisting of a solid form, a suspension and a solute.

20. The method of claim 7, wherein the step of contacting comprises spraying the silicone-based material with a preparation of the biological surfactant.

21. The method of claim 20, wherein the preparation of the biological surfactant is selected from the group consisting of a suspension, a solute and an aerosol.

22. The method of claim 7, wherein the step of contacting comprises condensing the biological surfactant on the silicone-based material.

23. The method of claim 7, wherein the step of contacting comprises depositing the biological surfactant on the silicone-based material.

24. A method for increasing evaporation from a silicone-based material, comprising the step of contacting a surface of the silicone-based material with a biological surfactant.

25. The method of claim 24, wherein the biological surfactant is selected from the group consisting of surfactant protein-A, surfactant protein-B, surfactant protein-C, and surfactant protein-D.

26. The method of claim 24, wherein the biological surfactant is a recombinant protein.

27. The method of claim 24, wherein the silicone-based material is selected from the group consisting of medical-grade tubing, a transdermal drug delivery patch, an implanted prosthesis and a contact lens.

28. The method of claim 24, wherein the silicone-based material is a contact lens.

29. The method of claim 24, wherein the biological surfactant is in solid form.

30. The method of claim 24, wherein the biological surfactant is in suspension.

31. The method of claim 24, wherein the biological surfactant is a solute.

32. The method of claim 24, wherein the biological surfactant is in the form of an aerosol.

33. The method of claim 24, wherein the biological surfactant is in a gaseous phase.

34. The method of claim 24, wherein the step of contacting comprises brushing the silicone-based material with a preparation of the biological surfactant.

35. The method of claim 24, wherein the step of contacting comprises dipping the silicone-based material in a preparation of the biological surfactant.

36. The method of claim 24, wherein the preparation of the biological surfactant is selected from the group consisting of a solid form, a suspension and a solute.

37. The method of claim 24, wherein the step of contacting comprises spraying the silicone-based material with a preparation of the biological surfactant.

38. The method of claim 37, wherein the preparation of the biological surfactant is selected from the group consisting of a suspension, a solute and an aerosol.

39. The method of claim 24, wherein the step of contacting comprises condensing the biological surfactant onto the silicone-based material.

40. The method of claim 24, wherein the step of contacting comprises depositing the biological surfactant onto the silicone-based material.

41. A composition comprising electrolytes and a biological surfactant.

42. The composition of claim 41, wherein the biological surfactant is selected from the group consisting of surfactant protein-A, surfactant protein-B, surfactant protein-C, and surfactant protein-D.

43. The composition of claim 41, wherein the biological surfactant is a recombinant protein.

44. A method for increasing expression of interleukin-8 during inflammation, comprising the step of contacting a cell with a surfactant protein.

45. The method of claim 44, wherein the inflammation results from ocular keratitis.

46. The method of claim 44, wherein the inflammation results from a stimulus.

47. The method of claim 46, wherein the stimulus is selected from the group consisting of a mechanical insult, ultraviolet radiation, a pathogen, a molecule derived from a pathogen, an antimicrobial peptide, and an object.

48. The method of claim 47, wherein the ultraviolet radiation has a wavelength selected from the group consisting of about 100 nanometers to about 280 nanometers, about 280 nanometers to about 315 nanometers, and about 315 nanometers to about 400 nanometers.

49. The method of claim 47, wherein the pathogen is selected from the group consisting of a bacterium, a fungus, and a virus.

50. The method of claim 49, wherein the bacterium is a gram-positive bacteria.

51. The method of claim 50, wherein the gram-positive bacteria is a species of Staphylococcus aureus.

52. The method of claim 47, wherein the molecule derived from a pathogen is selected from a group consisting of a molecule derived from a bacterium, a molecule derived from a fungus, and molecule derived from a virus.

53. The method of claim 52, wherein the molecule derived from the bacterium is a peptidoglycan.

54. The method of claim 53, wherein the bacterium is a gram-positive bacterium.

55. The method of claim 54, wherein the bacterium is a species of Staphylococcus aureus.

56. The method of claim 47, wherein the antimicrobial peptide is O-defensin.

57. The method of claim 47, wherein the object is debris.

58. The method of claim 57, wherein the debris is apoptotic debris.

59. The method of claim 57, wherein the debris is selected from the group consisting of bacterial debris and fungal debris.

60. The method of claim 44, wherein the cell is a mammalian cell.

61. The method of claim 60, wherein the cell is selected from the group consisting of a mouse cell, a rat cell, a ferret cell, a guinea-pig cell, a rabbit cell, a sheep cell, a goat cell, a pig cell, a cow cell, a dog cell, a cat cell, a monkey cell, a baboon cell, a chimpanzee cell, and a human cell.

62.-63. (canceled)

64. A method for decreasing expression of a biological surfactant, comprising the step of contacting a cell with an siRNA.

65. The method of claim 64, wherein the biological surfactant is selected from the group consisting of surfactant protein-A, surfactant protein-B, surfactant protein-C, and surfactant protein-D.

66. The method of claim 64, wherein the biological surfactant is a recombinant protein.

67. The method of claim 64, wherein the cell is a mammalian cell.

68. The method of claim 67, wherein the mammalian cell is selected from the group consisting of a mouse cell, a rat cell, a ferret cell, a guinea-pig cell, a rabbit cell, a sheep cell, a goat cell, a pig cell, a cow cell, a dog cell, a cat cell, a monkey cell, a baboon cell, a chimpanzee cell, and a human cell.

69. The method of claim 64, wherein the siRNA is a double-stranded siRNA.

70. The method of claim 69, wherein the 3′ end of one strand of the double-stranded RNA has a two-nucleotide overhang.

71. The method of claim 64, wherein a part of the nucleotide sequence of the siRNA is complementary to a part of a gene encoding a surfactant protein.

72. The method of claim 71, wherein the part of the nucleotide sequence of the siRNA is 19-25 nucleotides in length.

73. The method of claim 71, wherein the gene encoding the surfactant protein is selected from the gene encoding surfactant protein-A, the gene encoding surfactant protein-B, the gene encoding surfactant protein-C, and the gene encoding surfactant protein-D.
